(19) United States
(12) Patent Application Publication
(10) Pub. No.: US 2022/0040133 A1
(43) Pub. Date: Feb. 10, 2022

(54) ORAL THERAPY USING 6,8-BIS-BENZYLTHIO-OCTANOIC ACID

(71) Applicant: Rafael Pharmaceuticals, Inc., Cranbury, NJ (US)

(72) Inventors: Robert G.L. Shorr, Edison, NJ (US); Timothy S. Pardee, Cranbury, NJ (US); Lakmal Boteju, Kendall Park, NJ (US)

(21) Appl. No.: 17/414,404

(22) PCT Filed: Dec. 20, 2019
(86) PCT No.: PCT/US2019/067763

§ 371 Date: Jun. 16, 2021

Related U.S. Application Data
(60) Provisional application No. 62/834,478, filed on Apr. 16, 2019.
(60) Provisional application No. 62/782,938, filed on Dec. 20, 2018.

(51) Int. Cl. A61K 31/20; A61P 35/00; A61P 35/02

(52) U.S. Cl. CPC A61K 31/20; A61P 35/02; A61P 35/00

(57) ABSTRACT

The invention provides methods and compositions for treating a disease or disorder by orally administering to a patient in need thereof 6,8-bis-benzylthio-octanoic acid or a pharmaceutically acceptable salt thereof, optionally in combination with a second therapeutic agent.

### CROSS-REFERENCE TO RELATED APPLICATIONS

This application is the national stage application of International (PCT) Patent Application Serial No. PCT/US2019/067763, filed Dec. 20, 2019, which claims the benefit of and priority to U.S. Provisional Patent Application Ser. No. 62/782,938, filed Dec. 20, 2018 and U.S. Provisional Patent Application Ser. No. 62/834,478, filed Apr. 16, 2019; the contents of these applications are incorporated herein by reference.

### FIELD OF THE INVENTION

The invention provides methods and compositions for treating cancer by oral administration of 6,8-bis-benzylthio-octanoic acid or a pharmaceutically acceptable salt thereof.

### BACKGROUND

CPI-613 (6,8-bis-benzylthio-octanoic acid) is a first-in-class investigational small-molecule (lipoate analog), which targets the altered energy metabolism that is unique to many cancer cells. CPI-613 has been evaluated in multiple phase I, I/II, and II clinical studies, and has been granted orphan drug designation for the treatment of pancreatic cancer, acute myeloid leukemia (AML), peripheral T-cell lymphoma (PTCL), Burkitt lymphoma and myelodysplastic syndromes (MDS).

One limitation to the clinical utility of CPI-613 is its route of administration. CPI-613 is formulated as a 50 mg/mL solution in 1 M (150 mg/mL) aqueous triethanolamine, which is diluted with sterile 5% dextrose for injection prior to administration. For safety reasons, the resulting solution must be administered to a patient via a central venous catheter as an IV infusion over 30-120 minutes.

A need exists for an alternate method to safely and effectively administer CPI-613. The present invention addresses this need and provides other related advantages.

### SUMMARY

The invention provides methods and compositions for treating a disease or disorder by orally administering 6,8-bis-benzylthio-octanoic acid or a pharmaceutically acceptable salt thereof to a patient in need of such treatment. In certain embodiments, the disease or disorder is not a cancer of the pancreas or a cancer of the prostate. In certain embodiments, the disease or disorder may be a cancer, which may be, for example, relapsed or refractory. The cancer may be, for example, a lymphoma, leukemia, carcinoma, sarcoma, myeloma, brain or spinal cord cancer, melanoma, blastoma, germ cell tumor, cancer of the pancreas, or cancer of the prostate. In certain embodiments, the cancer is not a cancer of the pancreas or a cancer of the prostate. In certain embodiments, the cancer is relapsed or refractory Hodgkin lymphoma, including relapsed or refractory Hodgkin lymphoma in a patient who has failed brentuximab vedotin and a PD-1 inhibitor, relapsed or refractory T-cell non-Hodgkin lymphoma, relapsed or refractory Burkitt's lymphoma, or high-grade B-cell lymphoma with rearrangements of MYC and BCL2 and/or BCL6.

The invention further provides methods and compositions for treating a disease or disorder by orally administering 6,8-bis-benzylthio-octanoic acid or a pharmaceutically acceptable salt thereof to a patient in need of such treatment, provided that the disease or disorder is not prostate cancer, and further provided that when the disease or disorder is pancreatic cancer, the treatment does not further comprise administering to the patient a combination of (i) gemcitabine or a pharmaceutically acceptable salt thereof, and (ii) nab-paclitaxel. The invention further provides methods and compositions for treating a disease or disorder by orally administering 6,8-bis-benzylthio-octanoic acid or a pharmaceutically acceptable salt thereof to a patient in need of such treatment, provided that (a) the disease or disorder is not prostate cancer; (b) the treatment does not further comprise administering to the patient an autophagy inhibitor; and (c) when the disease or disorder is pancreatic cancer, the treatment does not further comprise administering to the patient a combination of (i) gemcitabine or a pharmaceutically acceptable salt thereof, and (ii) nab-paclitaxel. The invention further provides methods and compositions for treating cancer by orally administering 6,8-bis-benzylthio-octanoic acid or a pharmaceutically acceptable salt thereof to a patient in need of such treatment, provided that the cancer is not prostate cancer, and further provided that when the cancer is pancreatic cancer, the treatment does not further comprise administering to the patient a combination of (i) gemcitabine or a pharmaceutically acceptable salt thereof, and (ii) nab-paclitaxel. The invention further provides methods and compositions for treating cancer by orally administering 6,8-bis-benzylthio-octanoic acid or a pharmaceutically acceptable salt thereof to a patient in need of such treatment, provided that (a) the cancer is not prostate cancer, (b) the treatment does not further comprise administering to the patient an autophagy inhibitor; and (c) when the cancer is pancreatic cancer, the treatment does not further comprise administering to the patient a combination of (i) gemcitabine or a pharmaceutically acceptable salt thereof, and (ii) nab-paclitaxel. The invention further provides methods and compositions for treating cancer by orally administering 6,8-bis-benzylthio-octanoic acid or a pharmaceutically acceptable salt thereof to a patient in need of such treatment, provided that the cancer is not prostate cancer or pancreatic cancer. The invention further provides methods and compositions for treating cancer by orally administering 6,8-bis-benzylthio-octanoic acid or a pharmaceutically acceptable salt thereof to a patient in need of such treatment, provided that the cancer is not prostate cancer or pancreatic cancer, and further provided that the treatment does not further comprise administering to the patient an autophagy inhibitor. The cancer may be, for example, a lymphoma, leukemia, carcinoma, sarcoma, myeloma, brain or spinal cord cancer, melanoma, blastoma, or germ cell tumor.

The invention also provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that the patient is not in need of treatment for prostate cancer, and further provided that when the patient is in need of treatment for pancreatic cancer, the patient is not also administered a combination of (i) gemcitabine or a pharmaceutically acceptable salt thereof, and (ii) nab-paclitaxel for the treatment of the pancreatic cancer. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that (a) the patient is not in need of treatment for prostate cancer, (b) the patient is not also administered an autophagy inhibitor, and (c) when the patient is in need of treatment for pancreatic cancer, the patient is not also administered a combination with (i) gemcitabine or a pharmaceutically acceptable salt thereof, and (ii) nab-paclitaxel for the treatment of the pancreatic cancer. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that the patient is not in need of treatment for prostate cancer or pancreatic cancer. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that (a) the patient is not in need of treatment for prostate cancer or pancreatic cancer, and (b) the patient is not also administered an autophagy inhibitor.

The foregoing aspects of the invention are described in more detail, along with additional embodiments, in the detailed description below.

### BRIEF DESCRIPTION OF THE DRAWINGS

FIG. 1 depicts the anti-tumor efficacy of oral 6,8-bis-benzylthio-octanoic acid in human non-small cell lung cancer xenografts in mice.

FIG. 2 depicts the anti-tumor efficacy of oral 6,8-bis-benzylthio-octanoic acid in human pancreatic cancer xenografts in mice.

FIGS. 3A and 3B depict the treatment of MFL2 syngeneic tumors in C57Bl/6 mice with oral CPI-613 and either chloroquine or metformin, respectively.

FIG. 4 depicts the treatment of Baf3-P210 syngeneic tumors in Balb/c mice with oral CPI-613 and doxorubicin.

FIGS. 5A, 5B, and 5C present an X-ray powder diffraction pattern, differential scanning calorimetry thermogram, and proton nuclear magnetic resonance spectrum of CPI-613 piperazine material A.

FIGS. 6A, 6B, 6C, 6D, and 6E present an X-ray powder diffraction pattern, differential scanning calorimetry thermogram, proton nuclear magnetic resonance spectrum, thermogravimetric thermogram, and infrared spectroscopy of CPI-613 piperazine form B.

FIGS. 7A, 7B, 7C, 7D, and 7E present an X-ray powder diffraction pattern, thermogravimetric thermogram, proton nuclear magnetic resonance spectrum, differential scanning calorimetry thermogram, and infrared spectroscopy of CPI-613 piperazine material C.

FIGS. 8A, 8B, 8C, and 8D present an X-ray powder diffraction pattern, differential scanning calorimetry thermogram, proton nuclear magnetic resonance spectrum, and thermogravimetric thermogram of CPI-613 benzathine form A.

FIGS. 9A, 9B, 9C, 9D, and 9E present an X-ray powder diffraction pattern, differential scanning calorimetry thermogram, proton nuclear magnetic resonance spectrum, thermogravimetric thermogram, and infrared spectroscopy of CPI-613 benzathine material B.

FIGS. 10A, 10B, 10C, 10D, and 10E present an X-ray powder diffraction pattern, differential scanning calorimetry thermogram, proton nuclear magnetic resonance spectrum, infrared spectroscopy, and thermogravimetric thermogram of CPI-613 DL-lysine material A.

FIGS. 11A, 11B, 11C, 11D, and 11E present an X-ray powder diffraction pattern, differential scanning calorimetry thermogram, proton nuclear magnetic resonance spectrum, infrared spectroscopy, and thermogravimetric thermogram of CPI-613 triethanolamine form A.

FIG. 12 presents X-ray powder diffraction patterns solid amorphous dispersion formulations of 6,8-bis-benzylthio-octanoic acid with either Eudragit L100 or hydroxypropyl methylcellulose acetate succinate (HPMCAS-M) (top and middle diffraction patterns, respectively), and crystalline 6,8-bis-benzylthio-octanoic acid (bottom diffraction pattern).

### DETAILED DESCRIPTION

The invention provides methods and compositions for treating a disease or disorder in a patient in need thereof by orally administering to the patient a pharmaceutical composition comprising a therapeutically effective amount of 6,8-bis-benzylthio-octanoic acid or a pharmaceutically acceptable salt thereof, in order to treat the disease or disorder. The invention further provides methods and compositions for treating a disease or disorder in a patient in need thereof by orally administering to the patient a pharmaceutical composition comprising a therapeutically effective amount of 6,8-bis-benzylthio-octanoic acid or a pharmaceutically acceptable salt thereof, in order to treat the disease or disorder, provided that the disease or disorder is not prostate cancer, and further provided that when the disease or disorder is pancreatic cancer, the treatment does not further comprise administering to the patient a combination of (i) gemcitabine or a pharmaceutically acceptable salt thereof, and (ii) nab-paclitaxel. The invention further provides methods and compositions for treating a disease or disorder in a patient in need thereof by orally administering to the patient a pharmaceutical composition comprising a therapeutically effective amount of 6,8-bis-benzylthio-octanoic acid or a pharmaceutically acceptable salt thereof, in order to treat the disease or disorder, provided that (a) the disease or disorder is not prostate cancer, (b) the treatment does not further comprise administering to the patient an autophagy inhibitor, and (c) when the disease or disorder is pancreatic cancer, the treatment does not further comprise administering to the patient a combination of (i) gemcitabine or a pharmaceutically acceptable salt thereof, and (ii) nab-paclitaxel.

The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that the patient is not in need of treatment for prostate cancer, and further provided that when the patent is in need of treatment for pancreatic cancer, the patient is not also administered a combination of (i) gemcitabine or a pharmaceutically acceptable salt thereof, and (ii) nab-paclitaxel for the treatment of the pancreatic cancer. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that (a) the patient is not in need of treatment for prostate cancer, (b) the patient is not also administered an autophagy inhibitor, and (c) when the patient is in need of treatment for pancreatic cancer, the patient is not also administered a combination of (i) gemcitabine or a pharmaceutically acceptable salt thereof, and (ii) nab-paclitaxel for the treatment of the pancreatic cancer.

The practice of the present invention employs, unless otherwise indicated, conventional techniques of organic chemistry, pharmacology, and biochemistry. Such techniques are explained in the literature, such as “Comprehensive Organic Synthesis” (B. M. Trost & I. Fleming, eds., 1991-1992); which is incorporated by reference. Various aspects of the invention are set forth below in sections; however, aspects of the invention described in one particular section are not to be limited to any particular section.

### I. DEFINITIONS

To facilitate an understanding of the present invention, a number of terms and phrases are defined below.

The terms “a,” “an” and “the” as used herein mean “one or more” and include the plural unless the context is inappropriate

The term “6,8-bis-benzylthio-octanoic acid” refers to the compound known as devimistat or CPI-613, having the chemical structure

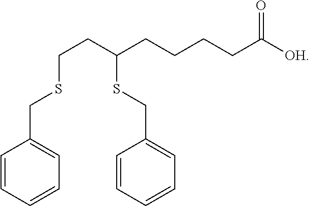 [SMILES: O=C(O)CCCCC(CCSCc1ccccc1)SCc1ccccc1]

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

As used herein, the term “autophagy inhibitor” refers to a compound capable of inhibiting any type of autophagy (e.g., macroautophagy, microautophagy, chaperone-mediated autophagy, mitophagy, or lipophagy) by any mechanism (e.g., by impacting formation of an autophagosome or its cargo). Examples of autophagy inhibitors include, but are not limited to, Mdivi-1, cyclosporine A, 4-aminoquinolines, 3-methyladenine (3-MA, CAS #5142-23-4), MHY1485 (CAS #326914-06-1SP600125), 3-methyl-6-(3-methylpiperidin-1-yl)-3H-purine, 6-chloro-N-(1-ethylpiperidin-4-yl)-1,2,3,4-tetrahydroacridin-9-amine, 4-(((1-(2-fluorophenyl)cyclopentyl)-amino)methyl)-2-((4-methylpiperazin-1-yl)methyl)phenol, 6-fluoro-N-[4-fluorobenzyl]quinazolin-4-amine, N-acetyl-L-cysteine, L-asparagine, N2,N4-dibenzylquinazoline-2,4-diamine, (2S,3S)-trans-Epoxysuccinyl-L-leucylamido-3-methylbutane ethyl ester, N-[6-(4-chlorophenoxy)hexyl]-N′-cyano-N″-4-pyridinyl-guanidine,leupeptin, 2-(4-morpholinyl)-8-phenyl-1(4H)-benzopyran-4-one, 4,6-Di-4-morpholinyl-N-(4-nitrophenyl)-1,3,5-triazin-2-amine, pepstatin A, 2-((5-Bromo-2-((3,4,5-trimethoxyphenyl)amino)pyrimidin-4-yl)oxy)-N-methylbenzamide, 6-Fluoro-N-[(4-fluorophenyl)methyl]-4-quinazolinamine, thapsigargin, amodiaquine, artemisinin, mefloquine, primaquine, piperaquine, quinacrine, U0126, 3-methyladenine, bafilomycin Al, chloroquine, hydroxychloroquine, verteporfin, LY294002, SB202190, SB203580, SC79, and wortmannin.

As used herein, the term “patient” refers to organisms to be treated by the methods of the present invention. Such organisms preferably include, but are not limited to, mammals (e.g., murines, simians, equines (horses), bovines (cattle), porcines, canines, felines, and the like). The term “patient” most preferably refers to humans.

“Therapeutically effective amount” refers to an amount of a compound sufficient to inhibit, halt, or cause an improvement in a disorder or condition being treated in a particular patient or patient population. For example, a therapeutically effective amount can be an amount of drug sufficient to slow the progression of a disease, or to prevent or delay its recurrence, such as maintenance treatment to prevent or delay relapse. A therapeutically effective amount can be determined experimentally in a laboratory or clinical setting, or may be the amount required by the guidelines of the United States Food and Drug Administration, or equivalent foreign agency, for the particular disease and patient being treated. It should be appreciated that determination of proper dosage forms, dosage amounts, and routes of administration is within the level of ordinary skill in the pharmaceutical and medical arts.

“Treatment” refers to the acute or prophylactic diminishment or alleviation of at least one symptom or characteristic associated or caused by a disorder being treated. For example, treatment can include diminishment of a symptom of a disorder or complete eradication of a disorder. As another example, treatment can include slowing the progression of a disease, or preventing or delaying its recurrence, such as maintenance treatment to prevent or delay relapse.

As used herein, the term “pharmaceutical composition” refers to the combination of an active agent with an excipient, inert or active, making the composition suitable for administration to a human.

The phrase “pharmaceutically acceptable” is employed herein to refer to those compounds, materials, compositions, and/or dosage forms which are, within the scope of sound judgment, suitable for use in contact with the tissues of human beings with acceptable toxicity, irritation, allergic response, and other problems or complications commensurate with a reasonable benefit/risk ratio.

As used herein, the term “pharmaceutically acceptable excipient” refers to any of the standard pharmaceutical excipients suitable for use in humans. For examples of excipients, see e.g., Martin, Remington's Pharmaceutical Sciences, 15th Ed., Mack Publ. Co., Easton, Pa. [1975].

As used herein, the term “pharmaceutically acceptable salt” refers to any salt (e.g., acid or base) of a compound of the present invention which is suitable for administration to a human. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Further examples of salts include salts made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, UIPAC, Wiley-VCH, P. H. Stahl, ed., the entire disclosure of which is incorporated by reference herein.

Further examples of salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Still other examples of salts include anions of the compounds of the present invention compounded with a suitable cation such as Na⁺, NH₄⁺, and NW₄⁺ (wherein W is a C₁₋₄ alkyl group), and the like. The term “alkyl” is art-recognized, and includes saturated aliphatic groups, including straight-chain alkyl groups and branched-chain alkyl groups.

In certain embodiments, the pharmaceutically acceptable salts are those prepared from the following acids: hydrochloric, hydrobromic, sulfuric, nitric, phosphoric, maleic, acetic, palicylic, p-toluene sulfonic, tartaric, citric, methane sulfonic, formic, malonic, succinic, naphthalene-2-sulfonic, and benzene sulfonic. In certain other embodiments, the pharmaceutically acceptable salts are alkaline metal or alkaline earth salts, such as sodium, potassium or calcium salts of a carboxylic acid group.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

As a general matter, compositions specifying a percentage are by weight unless otherwise specified. Further, if a variable is not accompanied by a definition, then the previous definition of the variable controls.

### II. THERAPEUTIC APPLICATIONS

The invention provides methods and compositions for treating a disease or disorder in a patient in need thereof by orally administering to the patient a pharmaceutical composition comprising a therapeutically effective amount of 6,8-bis-benzylthio-octanoic acid or a pharmaceutically acceptable salt thereof, in order to treat the disease or disorder. The invention further provides methods and compositions for treating a disease or disorder in a patient in need thereof by orally administering to the patient a pharmaceutical composition comprising a therapeutically effective amount of 6,8-bis-benzylthio-octanoic acid or a pharmaceutically acceptable salt thereof, in order to treat the disease or disorder, provided that the disease or disorder is not prostate cancer, and further provided that when the disease or disorder is pancreatic cancer, the treatment does not comprise administering to the patient a combination of (i) gemcitabine or a pharmaceutically acceptable salt thereof, and (ii) nab-paclitaxel. The invention further provides methods and compositions for treating a disease or disorder in a patient in need thereof by orally administering to the patient a pharmaceutical composition comprising a therapeutically effective amount of 6,8-bis-benzylthio-octanoic acid or a pharmaceutically acceptable salt thereof, in order to treat the disease or disorder, provided that (a) the disease or disorder is not prostate cancer; (b) the treatment does not further comprise administering to the patient an autophagy inhibitor; and (c) when the disease or disorder is pancreatic cancer, the treatment does not comprise administering to the patient a combination of (i) gemcitabine or a pharmaceutically acceptable salt thereof, and (ii) nab-paclitaxel. The invention further provides methods and compositions for treating a disease or disorder in a patient in need thereof by orally administering to the patient a pharmaceutical composition comprising a therapeutically effective amount of 6,8-bis-benzylthio-octanoic acid or a pharmaceutically acceptable salt thereof, in order to treat the disease or disorder, provided that the disease or disorder is not prostate cancer or pancreatic cancer. The invention further provides methods and compositions for treating a disease or disorder in a patient in need thereof by orally administering to the patient a pharmaceutical composition comprising a therapeutically effective amount of 6,8-bis-benzylthio-octanoic acid or a pharmaceutically acceptable salt thereof, in order to treat the disease or disorder, provided that (a) the disease or disorder is not prostate cancer or pancreatic cancer; and (b) the treatment does not further comprise administering to the patient an autophagy inhibitor.

The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that the patient is not in need of treatment for prostate cancer, and further provided that when the patient is need of treatment for pancreatic cancer, the patient is not also administered a combination of (i) gemcitabine or a pharmaceutically acceptable salt thereof, and (ii) nab-paclitaxel for the treatment of the pancreatic cancer. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that (a) the patient is not in need of treatment for prostate cancer, (b) the patient is not also administered an autophagy inhibitor, and (c) when the patient is need of treatment for pancreatic cancer, the patient is not also administered a combination of (i) gemcitabine or a pharmaceutically acceptable salt thereof, and (ii) nab-paclitaxel for the treatment of the pancreatic cancer. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that the patient is not in need of treatment for prostate cancer or pancreatic cancer. The invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, provided that (a) the patient is not in need of treatment for prostate cancer or pancreatic cancer, and (b) the patient is not also administered an autophagy inhibitor.

### Type of Disease or Disorder

In certain embodiments, the disease or disorder is associated with altered energy metabolism. In certain embodiments, the disease or disorder is a cancer. In certain embodiments, the disease or disorder is a cancer other than prostate cancer or pancreatic cancer. In certain embodiments, the disease or disorder is a myelodysplastic syndrome. In certain embodiments, the disease or disorder is Alzheimer's disease. In certain embodiments, the disease or disorder is diabetes. In certain embodiments, the disease or disorder is a microbial infection. In certain embodiments, the microbial infection is a bacterial infection, such as an infection by an Actinomyces, a Campylobacter (e.g., Campylobacter jejuni), an Escherichia (e.g., Escherichia coli), a Leptospira, a Pseudomonas (e.g., Pseudomonas aeruginosa), a Shigella (e.g., Shigella boydii), a Staphylococcus (e.g., Staphylococcal aureus), or a Streptococcus (e.g., Streptococcus pneumoniae) bacterium. In certain embodiments, the microbial infection is a yeast infection (e.g., a Candida) or fungal infection (e.g., a Cryptococcus). In certain embodiments, the microbial infection is a eukaryotic infection, e.g., by Cryptosporidium, Giardia, Leishmania, Neospora, Plasmodia, Toxoplasma, Trichomonas, or Trypanosoma. In certain embodiments, the disease or disorder is a hyperproliferative disease. In certain embodiments, the disease or disorder is psoriasis. In certain embodiments, the disease or disorder is a neuropathy. In certain embodiments, the disease or disorder is diabetic neuropathy.

Preferably, the disease or disorder is cancer. The method may be further characterized according to the severity or type of cancer. In certain embodiments, the cancer is Stage I or early stage cancer, in which the cancer is small and only in one area. In certain embodiments, the cancer is Stage II or III, in which the cancer is larger and has grown into nearby tissues or lymph nodes. In certain embodiments, the cancer Stage IV or advanced or metastatic, in which the cancer has spread to other parts of the body.

In certain embodiments, the cancer is Stage I lymphoma, in which the cancer is found in one lymph node region or the cancer has invaded one extralymphatic organ or site but not any lymph node regions. In certain embodiments, the cancer is Stage II lymphoma, in which the cancer is found in two or more lymph node regions on the same side of the diaphragm or the cancer involves one organ and its regional lymph nodes, with or without cancer in other lymph node regions on the same side of the diaphragm. In certain embodiments, the cancer is Stage III lymphoma, in which there is cancer in lymph nodes on both sides of the diaphragm. In certain embodiments, the cancer is Stage IV lymphoma, in which the cancer has spread one or more organs beyond the lymph nodes.

In certain embodiments, the cancer is progressive or refractory. In certain embodiments, the cancer is a metastatic. In certain embodiments, the cancer is recurrent or relapsed. In certain embodiments, the cancer is relapsed or refractory. In certain embodiments, the cancer is previously untreated. In certain embodiments, the cancer is previously untreated with systemic therapies. In certain embodiments, the cancer is previously untreated with systemic therapies or local treatment with chemoradiation. In certain embodiments, the patient has not received hematopoietic cell transplant. In certain embodiments, the patient has received hematopoietic cell transplant.

In certain embodiments, the cancer is a lymphoma. In certain embodiments, the cancer is a T-cell lymphoma. In certain embodiments, the cancer is a B-cell lymphoma. In certain embodiments, the cancer is a mantle cell lymphoma. In certain embodiments, the cancer is a leukemia. In certain embodiments, the cancer is an acute myeloid leukemia. In certain embodiments, the cancer is a carcinoma. In certain embodiments, the cancer is a sarcoma. In certain embodiments, the cancer is a myeloma. In certain embodiments, the cancer is a brain or spinal cord cancer. In certain embodiments, the cancer is a melanoma. In certain embodiments, the cancer is a blastoma. In certain embodiments, the cancer is a germ cell tumor. In certain embodiments, the disease or disorder is a cancer of the pancreas. In certain embodiments, the disease or disorder is not a cancer of the pancreas. In certain embodiments, the cancer is a metastatic pancreatic cancer. In certain embodiments, the cancer is a locally advanced pancreatic cancer. In certain embodiments, the cancer is a histologically or cytologically documented and measurable locally advanced pancreatic adenocarcinoma. In certain embodiments, the cancer is a histologically or cytologically documented and measurable metastatic pancreatic adenocarcinoma. In certain embodiments, the cancer is a histologically or cytologically documented and measurable locally advanced pancreatic adenocarcinoma that is previously untreated. In certain embodiments, the cancer is a histologically or cytologically documented and measurable metastatic pancreatic adenocarcinoma that is previously untreated. In certain embodiments, the cancer is a histologically or cytologically documented and measurable locally advanced pancreatic adenocarcinoma that is previously untreated with systemic therapies. In certain embodiments, the cancer is a histologically or cytologically documented and measurable metastatic pancreatic adenocarcinoma that is previously untreated with systemic therapies. In certain embodiments, the cancer is a histologically or cytologically documented and measurable locally advanced pancreatic adenocarcinoma that is previously untreated with systemic therapies or local treatment with chemoradiation. In certain embodiments, the cancer is a histologically or cytologically documented and measurable metastatic pancreatic adenocarcinoma that is previously untreated with systemic therapies or local treatment with chemoradiation. In certain embodiments, the cancer is a locally advanced pancreatic adenocarcinoma. In certain embodiments, the cancer is a metastatic pancreatic adenocarcinoma. In certain embodiments, the cancer is a locally advanced pancreatic adenocarcinoma that is previously untreated. In certain embodiments, the cancer is a metastatic pancreatic adenocarcinoma that is previously untreated. In certain embodiments, the cancer is a locally advanced pancreatic adenocarcinoma that is previously untreated with systemic therapies. In certain embodiments, the cancer is a metastatic pancreatic adenocarcinoma that is previously untreated with systemic therapies. In certain embodiments, the cancer is a locally advanced pancreatic adenocarcinoma that is previously untreated with systemic therapies or local treatment with chemoradiation. In certain embodiments, the cancer is a pancreatic adenocarcinoma that is previously untreated with systemic therapies or local treatment with chemoradiation. In certain embodiments, the cancer is a metastatic pancreatic cancer that is not concurrently being treated with a combination of (i) gemcitabine or a pharmaceutically acceptable salt thereof, and (ii) nab-paclitaxel. In certain embodiments, the cancer is a locally advanced pancreatic cancer that is not concurrently being treated with a combination of (i) gemcitabine or a pharmaceutically acceptable salt thereof, and (ii) nab-paclitaxel. In certain embodiments, the cancer is a histologically or cytologically documented and measurable locally advanced pancreatic adenocarcinoma that is not concurrently being treated with a combination of (i) gemcitabine or a pharmaceutically acceptable salt thereof, and (ii) nab-paclitaxel. In certain embodiments, the cancer is a histologically or cytologically documented and measurable metastatic pancreatic adenocarcinoma that is not concurrently being treated with a combination of (i) gemcitabine or a pharmaceutically acceptable salt thereof, and (ii) nab-paclitaxel. In certain embodiments, the cancer is a histologically or cytologically documented and measurable locally advanced pancreatic adenocarcinoma that is previously untreated and is not concurrently being treated with a combination of (i) gemcitabine or a pharmaceutically acceptable salt thereof, and (ii) nab-paclitaxel. In certain embodiments, the cancer is a histologically or cytologically documented and measurable metastatic pancreatic adenocarcinoma that is previously untreated and is not concurrently being treated with a combination of (i) gemcitabine or a pharmaceutically acceptable salt thereof, and (ii) nab-paclitaxel. In certain embodiments, the cancer is a histologically or cytologically documented and measurable locally advanced pancreatic adenocarcinoma that is previously untreated with systemic therapies and is not concurrently being treated with a combination of (i) gemcitabine or a pharmaceutically acceptable salt thereof, and (ii) nab-paclitaxel. In certain embodiments, the cancer is a histologically or cytologically documented and measurable metastatic pancreatic adenocarcinoma that is previously untreated with systemic therapies and is not concurrently being treated with a combination of (i) gemcitabine or a pharmaceutically acceptable salt thereof, and (ii) nab-paclitaxel. In certain embodiments, the cancer is a histologically or cytologically documented and measurable locally advanced pancreatic adenocarcinoma that is previously untreated with systemic therapies or local treatment with chemoradiation and is not concurrently being treated with a combination of (i) gemcitabine or a pharmaceutically acceptable salt thereof, and (ii) nab-paclitaxel. In certain embodiments, the cancer is a histologically or cytologically documented and measurable metastatic pancreatic adenocarcinoma that is previously untreated with systemic therapies or local treatment with chemoradiation and is not concurrently being treated with a combination of (i) gemcitabine or a pharmaceutically acceptable salt thereof, and (ii) nab-paclitaxel. In certain embodiments, the cancer is a locally advanced pancreatic adenocarcinoma that is not concurrently being treated with a combination of (i) gemcitabine or a pharmaceutically acceptable salt thereof, and (ii) nab-paclitaxel. In certain embodiments, the cancer is a metastatic pancreatic adenocarcinoma that is not concurrently being treated with a combination of (i) gemcitabine or a pharmaceutically acceptable salt thereof, and (ii) nab-paclitaxel. In certain embodiments, the cancer is a locally advanced pancreatic adenocarcinoma that is previously untreated and is not concurrently being treated with a combination of (i) gemcitabine or a pharmaceutically acceptable salt thereof, and (ii) nab-paclitaxel. In certain embodiments, the cancer is a metastatic pancreatic adenocarcinoma that is previously untreated and is not concurrently being treated with a combination of (i) gemcitabine or a pharmaceutically acceptable salt thereof, and (ii) nab-paclitaxel. In certain embodiments, the cancer is a locally advanced pancreatic adenocarcinoma that is previously untreated with systemic therapies and is not concurrently being treated with a combination of (i) gemcitabine or a pharmaceutically acceptable salt thereof, and (ii) nab-paclitaxel. In certain embodiments, the cancer is a metastatic pancreatic adenocarcinoma that is previously untreated with systemic therapies and is not concurrently being treated with a combination of (i) gemcitabine or a pharmaceutically acceptable salt thereof, and (ii) nab-paclitaxel. In certain embodiments, the cancer is a locally advanced pancreatic adenocarcinoma that is previously untreated with systemic therapies or local treatment with chemoradiation and is not concurrently being treated with a combination of (i) gemcitabine or a pharmaceutically acceptable salt thereof, and (ii) nab-paclitaxel. In certain embodiments, the cancer is a pancreatic adenocarcinoma that is previously untreated with systemic therapies or local treatment with chemoradiation and is not concurrently being treated with a combination of (i) gemcitabine or a pharmaceutically acceptable salt thereof, and (ii) nab-paclitaxel. In certain embodiments, the disease or disorder is a cancer of the prostate. In certain embodiments, the disease or disorder is not a cancer of the prostate. In certain embodiments, the cancer is a castration resistant prostate cancer. In certain embodiments, the disease or disorder is a cancer of the lung. In certain embodiments, the disease or disorder is a cancer of the colon. In certain embodiments, the disease or disorder is a cancer of the rectum. In certain embodiments, the disease or disorder is a colorectal cancer. In certain embodiments, the cancer is a neuroendocrine tumor. In certain embodiments, the cancer is a gastroenteropancreatic neuroendocrine tumor. In certain embodiments, the disease or disorder is a cancer of the liver. In certain embodiments, the disease or disorder is a cancer of uterus. In certain embodiments, the disease or disorder is a cancer of the cervix. In certain embodiments, the disease or disorder is a cancer of the bladder. In certain embodiments, the disease or disorder is a cancer of the kidney. In certain embodiments, the disease or disorder is a cancer of the breast. In certain embodiments, the disease or disorder is a cancer of the ovary.

In certain embodiments, the cancer is Burkitt's Lymphoma. In certain embodiments, the cancer is relapsed or refractory Burkitt's Lymphoma. In certain embodiments, the cancer is relapsed or refractory Burkitt's Lymphoma in which the patient has failed at least one previous line of therapy. In certain embodiments, the cancer is relapsed or refractory Burkitt's Lymphoma in which the patient has failed prior bone marrow transplant. In certain embodiments, the cancer is double hit diffuse large B cell lymphoma. In certain embodiments, the cancer is high-grade B cell lymphoma with rearrangements of MYC and BCL2 and/or BCL6 (DHL/THL). In certain embodiments, the cancer is Hodgkin lymphoma. In certain embodiments, the cancer is non-Hodgkin lymphoma. In certain embodiments, the cancer is T-cell non-Hodgkin lymphoma. In certain embodiments, the cancer is relapsed or refractory Hodgkin lymphoma. In certain embodiments, the cancer is relapsed or refractory non-Hodgkin lymphoma. In certain embodiments, the cancer is relapsed or refractory T-cell non-Hodgkin lymphoma. In certain embodiments, the cancer is Hodgkin lymphoma in which the patient has not received hematopoietic cell transplant. In certain embodiments, the cancer is Hodgkin lymphoma in which the patient has received hematopoietic cell transplant. In certain embodiments, the cancer is non-Hodgkin lymphoma in which the patient has not received hematopoietic cell transplant. In certain embodiments, the cancer is non-Hodgkin lymphoma in which the patient has received hematopoietic cell transplant. In certain embodiments, the cancer is T-cell non-Hodgkin lymphoma in which the patient has not received hematopoietic cell transplant. In certain embodiments, the cancer is T-cell non-Hodgkin lymphoma in which the patient has received hematopoietic cell transplant. In certain embodiments, the cancer is relapsed or refractory Hodgkin lymphoma in which the patient has not received hematopoietic cell transplant. In certain embodiments, the cancer is relapsed or refractory Hodgkin lymphoma in which the patient has received hematopoietic cell transplant. In certain embodiments, the cancer is relapsed or refractory non-Hodgkin lymphoma in which the patient has not received hematopoietic cell transplant. In certain embodiments, the cancer is relapsed or refractory Hodgkin lymphoma in which the patient has or has not received hematopoietic cell transplant. In certain embodiments, the cancer is relapsed or refractory Hodgkin lymphoma in which the patient has failed brentuximab vedotin and a PD-1 inhibitor. In certain embodiments, the cancer is relapsed or refractory Hodgkin lymphoma in which the patient has failed brentuximab vedotin and a PD-1 inhibitor and has received hematopoietic cell transplant. In certain embodiments, the cancer is relapsed or refractory Hodgkin lymphoma in which the patient has failed brentuximab vedotin and a PD-1 inhibitor and has not received hematopoietic cell transplant. In certain embodiments, the cancer is relapsed or refractory non-Hodgkin lymphoma in which the patient has received hematopoietic cell transplant. In certain embodiments, the cancer is relapsed or refractory T-cell non-Hodgkin lymphoma in which the patient has not received hematopoietic cell transplant. In certain embodiments, the cancer is relapsed or refractory T-cell non-Hodgkin lymphoma in which the patient has received hematopoietic cell transplant. In certain embodiments, the cancer is relapsed or refractory T-cell non-Hodgkin lymphoma in which the patient has or has not received hematopoietic cell transplant.

### General Aspects of Administering a Therapeutic Agent to a Patient

Generally, the therapeutic agent—i.e., 6,8-bis-benzylthio-octanoic acid or a pharmaceutically acceptable salt thereof—is delivered to the patient in a therapeutically effective amount, sufficient to treat the disease or disorder. The treatment may involve one or several administrations on one or more days, and the dosage may be adjusted by the individual practitioner to achieve a desired effect. Preferably, the dosage amount of the agent(s) used should be sufficient to interact primarily with disease cells, leaving normal cells comparatively unharmed.

The dosage amount may be administered in a single dose or in the form of individual divided doses, such as from one to four or more times per day. In certain embodiments, the daily dosage amount is administered in a single dose. In the event that the response in a patient is insufficient at a certain dose, higher doses (or effectively higher doses by a different, more localized delivery route) may be employed to the extent of patient tolerance.

For combination therapy, components in a combination therapy may be administered in a particular order and/or on the same or different days according to a treatment cycle. For example, in certain embodiments, at least one dose of the 6,8-bis-benzylthio-octanoic acid or a pharmaceutically acceptable salt thereof is administered to the patient prior to administering a second therapeutic agent, such as on an earlier day in a treatment cycle. In certain embodiments, active components of a combination therapy may be administered on the same day of a treatment cycle, for example being co-administered simultaneously. In certain embodiments, at least one dose of a second therapeutic agent is administered to the patient prior to administering the 6,8-bis-benzylthio-octanoic acid or a pharmaceutically acceptable salt thereof, such as on an earlier day in a treatment cycle. In certain embodiments, treatment cycles may be repeated one or more times in order to maximize benefit to the patient.

### 6,8-Bis-Benzylthio-Octanoic Acid and Pharmaceutically Acceptable Salts Thereof

In certain embodiments, the therapeutic agent is 6,8-bis-benzylthio-octanoic acid. In certain embodiments, the therapeutic agent is amorphous 6,8-bis-benzylthio-octanoic acid. In certain other embodiments, the therapeutic agent is a salt of 6,8-bis-benzylthio-octanoic acid.

Exemplary ion pairing agents that may be used to prepare pharmaceutically acceptable salts of 6,8-bis-benzylthio-octanoic acid include, for example, a tertiary amine (such as triethanolamine), a secondary amine, or a primary amine, such as diethanolamine, monoethanolamine, mefenamic acid and tromethamine, and combinations thereof. In certain embodiments, the therapeutic agent is a salt of 6,8-bis-benzylthio-octanoic acid and an organic Bronsted base. In certain embodiments, the therapeutic agent is a salt of 6,8-bis-benzylthio-octanoic acid with an amine compound. In certain embodiments, the therapeutic agent is a salt of 6,8-bis-benzylthio-octanoic acid with a monoalkylamine, dialkylamine, trialkylamine, amino-substituted aliphatic alcohol, hydroxymonoalkylamine, hydroxydialkylamine, hydroxytrialkylamine, amino-substituted heteroaliphatic alcohol, alkyldiamine, substituted alkyldiamine, or optionally substituted heteroaryl group containing at least one ring nitrogen atom. See, for example, Berge et al., “Pharmaceutical Salts,” J. of Pharmaceutical Science, 1977; 66:1-19.

In certain embodiments, the therapeutic agent is a salt of 6,8-bis-benzylthio-octanoic acid and polyethyleneimine, polyglutamic acid, ammonia, L-arginine, benethamine benzathine, betaine, calcium hydroxide, choline, deanol, diethanolamine(2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, or zinc hydroxide. In certain embodiments, the therapeutic agent is a salt of 6,8-bis-benzylthio-octanoic acid with diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, or ammonium hydroxide. In certain embodiments, the therapeutic agent is a salt of 6,8-bis-benzylthio-octanoic acid with an alkali metal hydroxide or alkaline earth metal hydroxide, such as, for example, cesium hydroxide.

In certain embodiments, the therapeutic agent is a salt of 6,8-bis-benzylthio-octanoic acid with a polymer-conjugated ion pairing agent which employs, without limitation, polyethylene glycol, polyethyleneimine, polyglutamic acid or a sugar-based polymer such as a dextran in combination with any of the above-noted ion pairing agents or any other known ion pairing agent. In certain embodiments, the therapeutic agent is a salt of 6,8-bis-benzylthio-octanoic acid with an ion pairing agent selected with guidance from Handbook of Pharmaceutical Salts Properties, Selection and Use, IUPAC, Wiley-VCH, P. H. Stahl, ed., the entire disclosure of which is incorporated by reference herein. Ion pairing agents of particular note therein include, without limitation, those listed in Table 5, p. 342.

In certain embodiments, the therapeutic agent is a triethanolamine salt of 6,8-bis-benzylthio-octanoic acid. In certain embodiments, the therapeutic agent is a piperazine salt of 6,8-bis-benzylthio-octanoic acid. In certain embodiments, the therapeutic agent is a benzathine salt of 6,8-bis-benzylthio-octanoic acid. In certain embodiments, the therapeutic agent is a DL-lysine salt of 6,8-bis-benzylthio-octanoic acid. In certain embodiments, the therapeutic agent is a choline, meglumine, tromethamine, L-arginine, L-lysine, potassium, sodium, calcium, or magnesium salt of 6,8-bis-benzylthio-octanoic acid. In certain embodiments, the therapeutic agent is a choline salt of 6,8-bis-benzylthio-octanoic acid. In certain embodiments, the therapeutic agent is a meglumine salt of 6,8-bis-benzylthio-octanoic acid. In certain embodiments, the therapeutic agent is a tromethamine salt of 6,8-bis-benzylthio-octanoic acid. In certain embodiments, the therapeutic agent is an L-arginine salt of 6,8-bis-benzylthio-octanoic acid. In certain embodiments, the therapeutic agent is a L-lysine salt of 6,8-bis-benzylthio-octanoic acid. In certain embodiments, the therapeutic agent is a potassium, sodium, calcium, or magnesium salt of 6,8-bis-benzylthio-octanoic acid. In certain embodiments, the therapeutic agent is a potassium salt of 6,8-bis-benzylthio-octanoic acid. In certain embodiments, the therapeutic agent is a sodium salt of 6,8-bis-benzylthio-octanoic acid. In certain embodiments, the therapeutic agent is a calcium salt of 6,8-bis-benzylthio-octanoic acid. In certain embodiments, the therapeutic agent is a magnesium salt of 6,8-bis-benzylthio-octanoic acid.

In certain embodiments, the therapeutic agent is a piperazine salt of 6,8-bis-benzylthio-octanoic acid, designated as CPI-613 piperazine material A. In certain embodiments, the CPI-613 piperazine material A exhibits an x-ray powder diffraction pattern having peaks at 3.22, 6.47, 9.72, 15.76, 16.34, 18.89, 19.43, 20.75, 21.00, 21.76, 22.96, 23.83, 25.12, 26.16, and 26.56 (±0.2°2θ). In certain embodiments, the CPI-613 piperazine material A exhibits an x-ray powder diffraction pattern having peaks at 3.22, 18.89, 19.43, 20.75, and 21.00 (±0.2°2θ).

In certain embodiments, the therapeutic agent is a piperazine salt of 6,8-bis-benzylthio-octanoic acid, designated as CPI-613 piperazine form B. In certain embodiments, the CPI-613 piperazine form B exhibits an x-ray powder diffraction pattern having peaks at 5.09, 7.30, 7.90, 8.16, 9.04, 9.62, 10.23, 10.83, 11.70, 12.27, 12.69, 13.61, 13.92, 14.68, 15.38, 15.88, 16.31, 16.92, 17.31, 17.51, 17.98, 18.62, 19.03, 19.35, 20.12, 20.60, 21.16, 21.40, 21.78, 22.24, 22.59, 23.12, 24.07, 24.92, 25.38, 26.35, 27.12, 27.60, and 28.02 (±0.2°2θ). In certain embodiments, the CPI-613 piperazine form B exhibits an x-ray powder diffraction pattern having peaks at 7.30, 15.88, 16.31, 16.92, 17.31, 19.03, 19.35, 20.60, 21.78, 22.59, 24.07, and 26.35 (±0.2°2θ). In certain embodiments, the CPI-613 piperazine form B exhibits an x-ray powder diffraction pattern having peaks at 15.88, 16.31, 16.92, 19.03, 19.35, 20.60, 21.78, and 22.59 (±0.2°2θ). In certain embodiments, the CPI-613 piperazine form B exhibits an x-ray powder diffraction pattern having peaks at 15.88, 16.31, 16.92, 19.03, 19.35, and 20.60 (±0.2°2θ).

In certain embodiments, the therapeutic agent is a piperazine salt of 6,8-bis-benzylthio-octanoic acid, designated as CPI-613 piperazine material C. In certain embodiments, the CPI-613 piperazine material C exhibits an x-ray powder diffraction pattern having peaks at 3.39, 10.30, 11.43, 11.81, 13.24, 13.78, 15.56, 15.83, 16.30, 16.93, 17.27, 17.67, 18.36, 18.93, 19.64, 20.73, 21.86, 22.44, 22.79, 23.21, 23.74, 25.62, 26.85, and 27.72 (±0.2°2θ). In certain embodiments, the CPI-613 piperazine material C exhibits an x-ray powder diffraction pattern having peaks at 15.56, 15.83, 17.67, 18.36, 18.93, 20.73, 22.44, and 25.62 (±0.2°2θ). In certain embodiments, the CPI-613 piperazine material C exhibits an x-ray powder diffraction pattern having peaks at 15.83, 18.36, 18.93, and 20.73 (±0.2°2θ).

In certain embodiments, the therapeutic agent is a benzathine salt of 6,8-bis-benzylthio-octanoic acid, designated as CPI-613 benzathine form A. In certain embodiments, the CPI-613 benzathine form A exhibits an x-ray powder diffraction pattern having peaks at 5.43, 6.16, 7.16, 9.12, 10.83, 11.10, 12.30, 13.68, 14.58, 15.71, 15.95, 16.25, 17.94, 18.27, 18.73, 19.08, 19.89, 20.19, 20.49, 21.73, 22.35, 22.68, 23.21, 23.67, 24.00, 24.52, 24.72, 24.99, 25.72, 26.23, 26.60, 27.09, and 28.06 (±0.2°2θ). In certain embodiments, the CPI-613 benzathine form A exhibits an x-ray powder diffraction pattern having peaks at 5.43, 10.83, 12.30, 15.95, 17.94, 18.73, 19.08, 21.73, and 22.35 (±0.2°2θ). In certain embodiments, the CPI-613 benzathine form A exhibits an x-ray powder diffraction pattern having peaks at 5.43, 18.73, and 21.73 (±0.2°2θ).

In certain embodiments, the therapeutic agent is a benzathine salt of 6,8-bis-benzylthio-octanoic acid, designated as CPI-613 benzathine material B. In certain embodiments, the CPI-613 benzathine material B exhibits an x-ray powder diffraction pattern having peaks at 7.48, 7.91, 12.69, 13.19, 14.58, 15.02, 15.47, 15.88, 16.14, 16.39, 16.66, 16.99, 17.23, 17.43, 18.04, 18.41, 18.90, 19.19, 19.45, 19.76, 20.07, 20.53, 20.74, 21.01, 21.33, 21.78, 22.02, 22.34, 22.63, 23.53, 23.82, 24.08, 24.41, 24.92, 25.07, 25.54, 25.64, 25.93, 26.38, 26.69, 27.07, 27.60, 27.93, 28.37, 29.06, and 29.70 (±0.2°2θ). In certain embodiments, the CPI-613 benzathine material B exhibits an x-ray powder diffraction pattern having peaks at 7.48, 16.14, 16.66, 16.99, 17.23, 17.43, 18.41, 18.90, 19.45, 19.76, 22.34, 23.53, 24.08, and 24.41 (±0.2°2θ). In certain embodiments, the CPI-613 benzathine material B exhibits an x-ray powder diffraction pattern having peaks at 7.48, 16.14, 16.99, 17.23, 17.43, 18.90, 19.45, 22.34, and 24.08 (±0.2°2θ). In certain embodiments, the CPI-613 benzathine material B exhibits an x-ray powder diffraction pattern having peaks at 16.14, 17.23, 17.43, 19.45, and 22.34 (±0.2°2θ).

In certain embodiments, the therapeutic agent is a DL-lysine salt of 6,8-bis-benzylthio-octanoic acid, designated as CPI-613 DL-lysine material A. In certain embodiments, the CPI-613 DL-lysine material A exhibits an x-ray powder diffraction pattern having peaks at 2.67, 5.50, 8.05, 8.27, 13.15, 13.73, 15.73, 16.13, 16.62, 18.98, 19.34, 19.74, 20.06, 21.19, 21.80, 22.50, 23.82, 24.17, 26.03, 26.41, and 27.00 (±0.2°2θ). In certain embodiments, the CPI-613 DL-lysine material A exhibits an x-ray powder diffraction pattern having peaks at 2.67, 8.05, 18.98, 19.34, and 21.19 (±0.2°2θ). In certain embodiments, the CPI-613 DL-lysine material A exhibits an x-ray powder diffraction pattern having peaks at 2.67 and 18.98 (±0.2°2θ).

In certain embodiments, the therapeutic agent is a triethanolamine salt of 6,8-bis-benzylthio-octanoic acid, designated as CPI-613 triethanolamine form A. In certain embodiments, the CPI-613 triethanolamine form A exhibits an x-ray powder diffraction pattern having peaks at 2.76, 5.54, 8.33, 11.14, 11.87, 13.11, 13.92, 14.79, 16.42, 16.73, 17.48, 18.07, 19.02, 19.52, 20.23, 20.79, 21.37, 21.98, 22.37, 22.77, 23.04, 23.27, 23.94, 25.01, 26.42, 27.34, 28.07, 28.42, and 28.97 (±0.2°2θ). In certain embodiments, the CPI-613 triethanolamine form A exhibits an x-ray powder diffraction pattern having peaks at 2.76, 13.11, 13.92, 16.42, 16.73, 19.52, 20.23, 21.37, 22.37, and 22.77 (±0.2°2θ). In certain embodiments, the CPI-613 triethanolamine form A exhibits an x-ray powder diffraction pattern having peaks at 13.92, 19.52, 20.23, 21.37, 22.37, and 22.77 (±0.2°2θ).

In certain embodiments, the therapeutic agent (6,8-bis-benzylthio-octanoic acid or a pharmaceutically acceptable salt thereof) has a purity of at least about 50% (w/w). In certain embodiments, the therapeutic agent has a purity of at least about 60% (w/w). In certain embodiments, the therapeutic agent has a purity of at least about 70% (w/w). In certain embodiments, the therapeutic agent has a purity of at least about 80% (w/w). In certain embodiments, the therapeutic agent has a purity of at least about 90% (w/w). In certain embodiments, the therapeutic agent has a purity of at least about 95% (w/w). In certain embodiments, the therapeutic agent has a purity of at least about 96% (w/w). In certain embodiments, the therapeutic agent has a purity of at least about 97% (w/w). In certain embodiments, the therapeutic agent has a purity of at least about 98% (w/w). In certain embodiments, the therapeutic agent has a purity of at least about 99% (w/w).

### Pharmaceutical Composition

Any pharmaceutical composition suitable for oral administration may be used in the present invention. In certain embodiments, the pharmaceutical composition is a dry oral dosage form. In certain embodiments, the pharmaceutical composition is an oral dosage form chosen from tablet, pill, capsule, caplet, powder, granule, solution, suspension, and gel. Oral dosage forms may include pharmaceutically acceptable excipients, such as carriers, diluents, stabilizers, plasticizers, binders, glidants, disintegrants, bulking agents, lubricants, plasticizers, colorants, film formers, flavoring agents, preservatives, dosing vehicles, and any combination of any of the foregoing. Pharmaceutically acceptable excipients are determined in part by the particular composition being administered, as well as by the particular dosing schedule. Accordingly, there is a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000).

The pharmaceutical composition will generally include at least one inert excipient. Excipients include pharmaceutically compatible binding agents, lubricants, wetting agents, disintegrants, and the like. Tablets, pills, capsules, troches and the like can contain any of the following excipients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain a liquid excipient such as a fatty oil. In addition, dosage unit forms can contain various other materials that modify the physical form of the dosage unit, for example, coatings of sugar, shellac, or enteric agents. Further, a syrup may contain, in addition to the active compounds, sucrose as a sweetening agent and certain preservatives, dyes, colorings, and flavorings. In certain embodiments, the pharmaceutical composition comprises an excipient in an amount of about 5% to about 99%, such as about 10% to about 85%, by weight of the composition, with the therapeutic agent comprising the remainder. In certain embodiments, pharmaceutically acceptable excipients comprise about 20% to about 80% of the total weight of the composition. In certain embodiments, the pharmaceutical composition comprises the therapeutic agent in an amount of at least about 40% by weight of the composition, with one or more excipients comprising the remainder. In certain embodiments, the pharmaceutical composition comprises the therapeutic agent in an amount of at least about 50% by weight of the composition. In certain embodiments, the pharmaceutical composition comprises the therapeutic agent in an amount of at least about 60% by weight of the composition. In certain embodiments, the pharmaceutical composition comprises the therapeutic agent in an amount of at least about 70% by weight of the composition. In certain embodiments, the pharmaceutical composition comprises the therapeutic agent in an amount of at least about 80% by weight of the composition. In certain embodiments, the pharmaceutical composition comprises the therapeutic agent in an amount of at least about 90% by weight of the composition.

Diluents for solid compositions include, but are not limited to, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Binders for solid pharmaceutical compositions include, but are not limited to, acacia, tragacanth, sucrose, glucose, alginic acid, carbomer (e.g. Carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch. In certain embodiments, the pharmaceutical composition comprises a binder in an amount of about 0.5% to about 25%, such as about 0.75% to about 15%, by weight of the composition. In certain embodiments, the pharmaceutical composition comprises a binder in an amount of about 1% to about 10% by weight of the composition.

The dissolution rate of a compacted solid pharmaceutical composition in a patient's stomach may be increased by the addition of a disintegrant to the composition. Disintegrants include, but are not limited to, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, powdered cellulose, pregelatinized starch, sodium alginate, sodium starch glycolate (e.g. EXPLOTAB®) and starch. In certain embodiments, the pharmaceutical composition comprises a disintegrant in an amount of about 0.2% to about 30%, such as about 0.2% to about 10%, by weight of the composition. In certain embodiments, the pharmaceutical composition comprises a disintegrant in an amount of about 0.2% to about 5% by weight of the composition.

The pharmaceutical composition optionally comprises one or more pharmaceutically acceptable wetting agents. Such wetting agents are preferably selected to maintain the API in close association with water, a condition that is believed to improve bioavailability of the composition. Non-limiting examples of surfactants that can be used as wetting agents include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene, caprylic/capric mono- and diglycerides (e.g., Labrasol™ of Gattefosse), polyoxyethylene castor oil and polyoxyethylene hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. In certain embodiments, the pharmaceutical composition comprises a wetting agent in an amount of about 0.25% to about 15%, such as about 0.4% to about 10%, by weight of the composition. In certain embodiments, the pharmaceutical composition comprises a wetting agent in an amount of about 0.5% to about 5% by weight of the composition. In certain embodiments, the pharmaceutical composition comprises a wetting agent that is an anionic surfactant. In certain embodiments, the pharmaceutical composition comprises sodium lauryl sulfate as a wetting agent. In certain embodiments, the pharmaceutical composition comprises sodium lauryl sulfate in an amount of about 0.25% to about 7%, such as about 0.4% to about 4%, by weight of the composition. In certain embodiments, the pharmaceutical composition comprises sodium lauryl sulfate in an amount of about 0.5% to about 2% by weight of the composition.

Lubricants (e.g., anti-adherents or glidants) can be added to improve the flow properties of solid compositions and/or to reduce friction between the composition and equipment during compression of tablet formulations. Excipients that may function as lubricants include, but are not limited to, colloidal silicon dioxide, magnesium trisilicate, powdered cellulose, starch, talc and tribasic calcium phosphate. Suitable lubricants further include glyceryl behapate (e.g., Compritol™ 888 of Gattefosse); stearic acid and salts thereof, including magnesium, calcium and sodium stearates; zinc stearate; glyceryl monostearate; glyceryl palmitostearate; hydrogenated castor oil; hydrogenated vegetable oils (e.g., Sterotex™ of Abitec); waxes; boric acid; sodium benzoate; sodium acetate; sodium stearyl fumarate; sodium fumarate; sodium chloride; DL-leucine; PEG (e.g., Carbowax™ 4000 and Carbowax™ 6000 of the Dow Chemical Company); sodium oleate; sodium lauryl sulfate; and magnesium lauryl sulfate. In certain embodiments, the pharmaceutical compositions comprises a lubricant in an amount of about 0.1% to about 10%, such as about 0.2% to about 8%, by weight of the composition. In certain embodiments, the pharmaceutical composition comprises a lubricant in an amount of about 0.25% to about 5% by weight of the composition. In certain embodiments, the pharmaceutical composition comprises magnesium stearate as a lubricant. In certain embodiments, the pharmaceutical composition comprises colloidal silicon dioxide. In certain embodiments, the pharmaceutical composition comprises talc. In certain embodiments, the composition comprises magnesium stearate or talc in an amount of about 0.5% to about 2% by weight of the composition.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level.

Selection of excipients and the amounts to use may be readily determined by formulation scientists based upon experience and consideration of standard procedures and reference works in the field. The solid compositions of the present invention include powders, granulates, aggregates and compacted compositions. The dosages may be conveniently presented in unit dosage form and prepared by any of the methods well-known in the pharmaceutical arts. Dosage forms include solid dosage forms like tablets, pills, powders, caplets, granules, capsules, sachets, troches and lozenges. In certain embodiments, the pharmaceutical composition is a tablet. In certain embodiments, the pharmaceutical composition is a spray-dried dispersion. In certain embodiments, the pharmaceutical composition is a spray-dried dispersion comprising at least one polymer chosen from polyacrylate, polymethacrylate, poly(vinylpyrrolidone), hydroxypropyl methyl cellulose (HPMC), cellulose acetate phthalate (CAP), and hydroxypropyl methylcellulose acetate succinate (HPMCAS-M). In certain embodiments, the pharmaceutical composition is a spray-dried dispersion comprising at least one polymer chosen from Eudragit L100, poly(vinylpyrrolidone), hydroxypropyl methyl cellulose (HPMC), cellulose acetate phthalate (CAP), and hydroxypropyl methylcellulose acetate succinate (HPMCAS-M). In certain embodiments, the pharmaceutical composition is a spray-dried dispersion comprising at least one polymer chosen from Eudragit L100, poly(vinylpyrrolidone) viscosity grade K30 (PVP K30), hydroxypropyl methyl cellulose (HPMC), cellulose acetate phthalate (CAP), and hydroxypropyl methylcellulose acetate succinate (HPMCAS-M). In certain embodiments, the pharmaceutical composition is a spray-dried dispersion comprising at least one polymer chosen from Eudragit L100 and hydroxypropyl methylcellulose acetate succinate (HPMCAS-M). In certain embodiments, the pharmaceutical composition is a spray-dried dispersion comprising Eudragit L100. In certain embodiments, the pharmaceutical composition is a spray-dried dispersion comprising hydroxypropyl methylcellulose acetate succinate (HPMCAS-M).

The formulations of the invention may be buffered by the addition of suitable buffering agents.

In certain embodiments, the pharmaceutical composition of the present invention is a unit dose composition. In certain embodiments, the pharmaceutical composition contains about 1 mg to about 5000 mg of the therapeutic agent. In certain embodiments, the pharmaceutical composition contains about 100 mg to about 3000 mg of the therapeutic agent. In certain embodiments, the pharmaceutical composition contains about 200 mg to about 2000 mg of the therapeutic agent. In certain embodiments, the pharmaceutical composition contains about 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, or 3000 mg of therapeutic agent. In certain embodiments, the pharmaceutical composition contains about 300 mg, 500 mg, 700 mg, or 1000 mg of the therapeutic agent.

In certain embodiments, the pharmaceutical composition of the present invention comprises an emulsion, particle, or gel as described in U.S. Pat. No. 7,220,428. In certain embodiments, the pharmaceutical composition is a solid or liquid formulation having from about 0.1% to about 75% w/w lipids or fatty acid components. In certain embodiments, the formulation contains about 0.1% to about 15% w/v lipids and fatty acid components. In certain embodiments, the fatty acid component comprises saturated or unsaturated C4, C5, C6, C7, C8, C9, C10, C11, or C12 fatty acids and/or salts of such fatty acids. Lipids may include cholesterol and analogs thereof.

### Dosing Amounts & Regimens

The therapeutic agent may be orally administered to the patient in any suitable dose according to any suitable schedule. The dose and schedule will vary based on, e.g., the condition being treated and whether another therapeutic agent will be administered in combination, and can be readily determined by those of ordinary skill in the art in view of the guidance provided herein. In certain embodiments, the dose and schedule is adapted based on the dose and schedule used to effectively treat a disease or disorder intravenously with 6,8-bis-benzylthio-octanoic acid or a pharmaceutically acceptable salt thereof. In certain embodiments, the dose is the maximum tolerated dose.

An advantage of the present invention is that oral dosing permits substantially increased dosing flexibility as compared to the IV dosing of the prior art. In the prior art, 6,8-bis-benzylthio-octanoic acid is formulated as a 50 mg/mL solution in 1 M (150 mg/mL) aqueous triethanolamine, which is diluted from 50 mg/mL to as low as 4 mg/mL (e.g., 12.5 mg/mL) with sterile 5% dextrose for injection (D5W) prior to administration as an IV infusion over 30-120 minutes via a central venous catheter. Such an infusion is inconvenient for patients and effectively precludes regimens involving frequent, prolonged dosing. Since the half-life of bis-benzylthio-octanoic acid after IV dosing is only about 1-2 hours (Pardee, T. S. et al., Clin Cancer Res. 2014, 20, 5255-64), more frequent and/or prolonged dosing could advantageously be used to increase the patient's exposure to the bis-benzylthio-octanoic acid.

For example, in a recent phase I study 11 of 18 patients with metastatic pancreatic adenocarcinoma achieved an objective response when treated intravenously with 500 mg/m² 6,8-bis-benzylthio-octanoic acid (the maximum tolerated dose) on days 1 and 3 of a two week cycle, combined with modified FOLFIRINOX on day 1 of the cycle (oxaliplatin at 65 mg/m², leucovorin at 400 mg/m², irinotecan at 140 mg/m², and fluorouracil 400 mg/m² bolus followed by 2400 mg/m² over 46 h) and Neulasta (pegfilgrastim) on day 4 of the cycle (Alistar A. et al., Lancet Onol. 2017, 18, 770-78, incorporated herein by reference). According to the present invention, patients with metastatic pancreatic adenocarcinoma could be treated with modified FOLIFIRINOX on day 1 of a two week cycle as in the Alistar phase I study, but the practitioner would have flexibility with respect to the 6,8-bis-benzylthio-octanoic acid dose and schedule. The 6,8-bis-benzylthio-octanoic acid could be orally administered in a single daily dose on days 1 and 3 of the two week cycle as in the phase I study. Alternatively, the 6,8-bis-benzylthio-octanoic acid could be administered in two or more (e.g., three, four, or five) divided doses. The single or divided doses could be administered on days 1 and 3 of the cycle or different days of the cycle other than or in addition to days 1 and/or 3, up to and including every day.

In another phase I study, intravenous 6,8-bis-benzylthio-octanoic acid showed efficacy in patients with myelodysplastic syndrome (MDS), acute myeloid leukemia (AML), Burkitt lymphoma, and cutaneous T-cell lymphoma (CTCL) when administered on days 1 and 4 of weeks 1, 2, and 3 of a 4 week cycle at a daily dose of 840 mg/m² to 2940 mg/m² (Pardee, T. et al., Clin. Cancer Res. 2014, 20, 5255-64, incorporated herein by reference). According to the present invention, patients with MDS, AML, Burkitt lymphoma, or CTCL may be treated orally with 6,8-bis-benzylthio-octanoic acid according to the same or different schedule. The 6,8-bis-benzylthio-octanoic acid could be orally administered in a single daily dose on days 1 and 4 of weeks 1, 2, and 3 of a 4 week cycle as in the Pardee phase I study. Alternatively, the 6,8-bis-benzylthio-octanoic acid could be administered in two or more (e.g., three, four, or five) divided doses on those days or different days of the cycle other than or in addition to days 1 and 3, up to and including every day.

In another phase I study, intravenous 6,8-bis-benzylthio-octanoic acid showed efficacy in patients with relapsed or refractory T-cell lymphoma when administered on days 1-4, 8, 11, 15, and 18 of a 4 week cycle at a daily dose of 2000 mg/m² to 2750 mg/m², combined with bendamustine (90 mg/m²) on days 4 and 5 of the 4 week cycle (Lamar Z. et al., Blood 2016, 128, 4163, incorporated herein by reference). According to the present invention, patients with T-cell lymphoma may be treated with bendamustine on days 4 and 5 of a 4 week cycle as in the Lamar phase I study, but the 6,8-bis-benzylthio-octanoic acid could be administered according to the same or different schedule. The 6,8-bis-benzylthio-octanoic acid could be orally administered in a single daily dose on days 1-4, 8, 11, 15, and 18 of a 4 week cycle as in the Lamar phase I study. Alternatively, the 6,8-bis-benzylthio-octanoic acid could be administered in two or more (e.g., three, four, or five) divided doses. The single or divided doses could be administered on days 1-4, 8, 11, 15, and 18 of a 4 week cycle, or on different days of the cycle other than or in addition to days 1-4, 8, 11, 15, and 18, up to and including every day.

Another advantage of oral dosing is that it makes maintenance therapy feasible. For example, a patient who is treated successfully with first line therapy—with or without 6,8-bis-benzylthio-octanoic acid—and whose cancer is in partial or complete remission, may be treated orally with 6,8-bis-benzylthio-octanoic acid or a pharmaceutically acceptable salt thereof on a chronic basis in order to delay or prevent recurrence. The maintenance treatment may involve, for example, one, two, three, four, or five doses per day of 6,8-bis-benzylthio-octanoic acid or a pharmaceutically acceptable salt thereof on a regular basis, such as daily or weekly. In certain embodiments, the maintenance therapy is for the treatment of pancreatic cancer. In certain embodiments, the maintenance therapy is for the treatment of pancreatic cancer and the patient is not further administered gemcitabine and nab-paclitaxel. In certain embodiments, the maintenance therapy is for the treatment of pancreatic cancer and the patient is not further administered gemcitabine or nab-paclitaxel.

In certain embodiments, the 6,8-bis-benzylthio-octanoic acid or pharmaceutically acceptable salt thereof is orally administered at a dose of about 1 mg to about 10,000 mg on each day it is administered. The daily dose may be administered in one dose or divided into two or more doses, such as three, four, or five doses. In certain embodiments, the daily dose is about 10 mg to about 7,500 mg. In certain embodiments, the daily dose is about 100 mg to about 5,000 mg. In certain embodiments, the daily dose is about 200 mg to about 4,000 mg. In certain embodiments, the daily dose is about 300 mg to about 3,000 mg. In certain embodiments, the daily dose is about 400 mg to about 2,500 mg. In certain embodiments, the daily dose is about 500 mg to about 2,000 mg. In certain embodiments, the daily dose is about 100 mg, 200 mg, 300 mg, 400 mg, 500 mg, 600 mg, 700 mg, 800 mg, 900 mg, 1,000 mg, 1,250 mg, 1,500 mg, 1,750 mg, 2,000 mg, 2,500 mg, 3,000 mg, 3,500 mg, 4,000 mg, 4,500 mg, 5,000 mg, 6,000 mg, 7,000 mg, 8,000 mg, 9,000 mg, or 10,000 mg. In certain embodiments, each dose of 6,8-bis-benzylthio-octanoic acid or pharmaceutically acceptable salt thereof is about 0.5 g to 1.5 g, and is administered once, twice, three times, four times, or five times daily. In certain embodiments, each dose of 6,8-bis-benzylthio-octanoic acid or pharmaceutically acceptable salt thereof is about 0.5 g to 1.5 g, and is administered once daily. In certain embodiments, each dose of 6,8-bis-benzylthio-octanoic acid or pharmaceutically acceptable salt thereof is about 0.5 g to 1.5 g, and is administered twice daily. In certain embodiments, each dose of 6,8-bis-benzylthio-octanoic acid or pharmaceutically acceptable salt thereof is about 0.5 g to 1.5 g, and is administered three times daily. In certain embodiments, each dose of 6,8-bis-benzylthio-octanoic acid or pharmaceutically acceptable salt thereof is about 0.5 g to 1.5 g, and is administered four times daily. In certain embodiments, each dose of 6,8-bis-benzylthio-octanoic acid or pharmaceutically acceptable salt thereof is about 0.5 g to 1.5 g, and is administered five times daily. In certain embodiments, each dose of 6,8-bis-benzylthio-octanoic acid or pharmaceutically acceptable salt thereof is about 1 g, and is administered once, twice, three times, four times, or five times daily. In certain embodiments, each dose of 6,8-bis-benzylthio-octanoic acid or pharmaceutically acceptable salt thereof is about 1 g, and is administered once daily. In certain embodiments, each dose of 6,8-bis-benzylthio-octanoic acid or pharmaceutically acceptable salt thereof is about 1 g, and is administered twice daily. In certain embodiments, each dose of 6,8-bis-benzylthio-octanoic acid or pharmaceutically acceptable salt thereof is about 1 g, and is administered three times daily. In certain embodiments, each dose of 6,8-bis-benzylthio-octanoic acid or pharmaceutically acceptable salt thereof is about 1 g, and is administered four times daily. In certain embodiments, each dose of 6,8-bis-benzylthio-octanoic acid or pharmaceutically acceptable salt thereof is about 1 g, and is administered five times daily.

In certain embodiments, a dosing cycle is repeated at least once. In certain embodiments, the method of the present invention comprises treatment with two cycles or more. In certain embodiments, the method of the present invention comprises treatment with three cycles or more. In certain embodiments, the method of the present invention comprises treatment with four cycles or more. In certain embodiments, the method of the present invention comprises treatment with five cycles or more. In certain embodiments, the method of the present invention comprises treatment with six cycles or more. In certain embodiments, the method of the present invention comprises treatment with seven cycles or more. In certain embodiments, the method of the present invention comprises treatment with eight cycles or more. In certain embodiments, the method of the present invention comprises treatment with nine cycles or more. In certain embodiments, the method of the present invention comprises treatment with ten cycles or more. In certain embodiments, the method of the present invention comprises regular treatment with 6,8-bis-benzylthio-octanoic acid or a pharmaceutically acceptable salt thereof, including on a daily or weekly basis, for an extended period of time, such as at least one month, six months, one year, two years, three years, or longer.

### Second Therapeutic Agent

In certain embodiments, the method of the present invention further comprises administration of a therapeutically effective amount of a second therapeutic agent. For example, the present invention provides a method for treating a disease or disorder in a patient in need thereof, comprising the steps of (a) orally administering to the patient a pharmaceutical composition comprising a therapeutically effective amount of 6,8-bis-benzylthio-octanoic acid or a pharmaceutically acceptable salt thereof, and (b) administering to the patient a therapeutically effective amount of a second therapeutic agent, in order to treat the disease or disorder. The present invention provides a method for treating a disease or disorder in a patient in need thereof, comprising the steps of (a) orally administering to the patient a pharmaceutical composition comprising a therapeutically effective amount of 6,8-bis-benzylthio-octanoic acid or a pharmaceutically acceptable salt thereof, and (b) administering to the patient a therapeutically effective amount of a second therapeutic agent, in order to treat the disease or disorder, provided that the second therapeutic agent is not an autophagy inhibitor and the treatment does not comprise administering to the patient an autophagy inhibitor. The present invention further provides a method for treating a disease or disorder in a patient in need thereof, comprising the steps of (a) orally administering to the patient a pharmaceutical composition comprising a therapeutically effective amount of 6,8-bis-benzylthio-octanoic acid or a pharmaceutically acceptable salt thereof, and (b) administering to the patient a therapeutically effective amount of a second therapeutic agent, in order to treat the disease or disorder, provided that the disease or disorder is not prostate cancer, and further provided that when the disease or disorder is pancreatic cancer, the patient is not further administered a combination of (i) gemcitabine or a pharmaceutically acceptable salt thereof, and (ii) nab-paclitaxel. The present invention further provides a method for treating a disease or disorder in a patient in need thereof, comprising the steps of (a) orally administering to the patient a pharmaceutical composition comprising a therapeutically effective amount of 6,8-bis-benzylthio-octanoic acid or a pharmaceutically acceptable salt thereof, and (b) administering to the patient a therapeutically effective amount of a second therapeutic agent, in order to treat the disease or disorder, provided that (a) the disease or disorder is not prostate cancer; (b) the second therapeutic agent is not an autophagy inhibitor and the treatment does not comprise administering to the patient an autophagy inhibitor; and (c) when the disease or disorder is pancreatic cancer, the treatment does not further comprise administering to the patient a combination of (i) gemcitabine or a pharmaceutically acceptable salt thereof, and (ii) nab-paclitaxel. In certain embodiments, the present invention provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, and further administering to the patient a therapeutically effective amount of a second therapeutic agent. The present invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, and further administering to the patient a therapeutically effective amount of a second therapeutic agent, provided that the patient is not in need of treatment for prostate cancer, and further provided that when the patient is in need of treatment for pancreatic cancer, the patient is not also administered a combination of (i) gemcitabine or a pharmaceutically acceptable salt thereof, and (ii) nab-paclitaxel for the treatment of the pancreatic cancer. The present invention further provides a method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof, and further administering to the patient a therapeutically effective amount of a second therapeutic agent, provided that (a) the patient is not in need of treatment for prostate cancer, (b) the patient is not also administered an autophagy inhibitor, and (c) when the patient is in need of treatment for pancreatic cancer, the patient is not also administered a combination of (i) gemcitabine or a pharmaceutically acceptable salt thereof, and (ii) nab-paclitaxel for the treatment of the pancreatic cancer.

In certain embodiments, the second therapeutic agent is chemotherapeutic agent. In certain embodiments, the second therapeutic agent is bendamustine or a pharmaceutically acceptable salt thereof. In certain embodiments, the second therapeutic agent is bendamustine hydrochloride, e.g., when the disease or disorder is a lymphoma, such as Hodgkin lymphoma or non-Hodgkin lymphoma, including T-cell non-Hodgkin lymphoma. In certain embodiments, the second therapeutic agent is a combination of oxaliplatin, leucovorin, irinotecan, and fluorouracil, e.g., when the disease or disorder is pancreatic cancer. In certain embodiments, the second therapeutic agent is a combination of gemcitabine and nab-paclitaxel, e.g., when the disease or disorder is pancreatic cancer. In certain other embodiments, the second therapeutic agent is not a combination of gemcitabine and nab-paclitaxel, e.g., when the disease or disorder is pancreatic cancer. In certain embodiments, the second therapeutic agent is docetaxel, e.g., when the disease or disorder is prostate cancer. In certain other embodiments, the second therapeutic agent is not docetaxel, e.g., when the disease or disorder is prostate cancer. In certain embodiments, the second therapeutic agent is an autophagy inhibitor. In certain embodiments, the second therapeutic agent is not an autophagy inhibitor.

The second therapeutic agent may be administered according to any suitable schedule at any suitable dose. Appropriate doses and schedules for various diseases and disorders are known in the art and can be adapted to use with oral 6,8-bis-benzylthio-octanoic acid without undue experimentation.

Treatment Efficacy and Safety

The therapeutic method of the present invention may be further characterized by the efficacy and safety of the treatment. Preferably, the method provides an acceptable safety profile, with the benefit of treatment outweighing the risk. When tested in a phase II or phase III clinical trial of at least 10 patients with cancer, the method of the present invention preferably provides an overall response rate of at least about 10%, a duration of response of at least about 1 month, progression-free survival (PFS) of at least about 1 month, and/or overall survival (OS) of at least about 1 month. Preferably, the phase II or phase III clinical trial comprises at least 15 patients. More preferably, the phase II or phase III clinical trial comprises at least 20 patients. More preferably, the phase II or phase III clinical trial comprises at least 25 patients. More preferably, the phase II or phase III clinical trial comprises at least 50 patients. More preferably, the phase II or phase III clinical trial comprises at least 100 patients. More preferably, the phase II or phase III clinical trial comprises at least 200 patients. More preferably, the phase II or phase III clinical trial comprises at least 300 patients. More preferably, the phase II or phase III clinical trial comprises at least 400 patients. More preferably, the phase II or phase III clinical trial comprises at least 500 patients. Preferably, the method of the present invention provides an overall response rate of at least about 20% in patients. More preferably, the method of the present invention provides an overall response rate of at least about 30%. More preferably, the method of the present invention provides an overall response rate of at least about 40%. More preferably, the method of the present invention provides an overall response rate of at least about 50%. More preferably, the method of the present invention provides an overall response rate of at least about 60%. More preferably, the method of the present invention provides an overall response rate of at least about 70%. More preferably, the method of the present invention provides an overall response rate of at least about 80%. More preferably, the method of the present invention provides an overall response rate of at least about 90%. Preferably, the method of the present invention provides a duration of response, PFS, and/or OS of at least about 2 months. Preferably, the method of the present invention provides a duration of response, PFS, and/or OS of at least about 3 months. Preferably, the method of the present invention provides a duration of response, PFS, and/or OS of at least about 4 months. Preferably, the method of the present invention provides a duration of response, PFS, and/or OS of at least about 5 months. Preferably, the method of the present invention provides a duration of response, PFS, and/or OS of at least about 6 months. Preferably, the method of the present invention provides a duration of response, PFS, and/or OS of at least about 7 months. Preferably, the method of the present invention provides a duration of response, PFS, and/or OS of at least about 8 months. Preferably, the method of the present invention provides a duration of response, PFS, and/or OS of at least about 9 months. Preferably, the method of the present invention provides a duration of response, PFS, and/or OS of at least about 10 months. Preferably, the method of the present invention provides a duration of response, PFS, and/or OS of at least about 11 months. Preferably, the method of the present invention provides a duration of response, PFS, and/or OS of at least about 12 months. Preferably, the method of the present invention provides a duration of response, PFS, and/or OS of at least about 14 months. Preferably, the method of the present invention provides a duration of response, PFS, and/or OS of at least about 16 months. Preferably, the method of the present invention provides a duration of response, PFS, and/or OS of at least about 18 months. Preferably, the method of the present invention provides a duration of response, PFS, and/or OS of at least about 20 months. Preferably, the method of the present invention provides a duration of response, PFS, and/or OS of at least about 24 months. In certain embodiments, the overall response rate, duration of response, and progression-free survival mentioned above are measured in a phase II clinical trial. In certain embodiments, the overall response rate, duration of response, and progression-free survival mentioned above are measured in a phase III clinical trial.

### Patients for Treatment

The therapeutic methods may be further characterized according to the patient to be treated. Preferably, the patient is a human. In certain embodiments, the patient is an adult human.

### EQUIVALENTS

The description above describes multiple aspects and embodiments of the invention, including therapeutic applications, treatment methods, and pharmaceutical compositions. The patent application specifically contemplates all combinations and permutations of the aspects and embodiments.

### III. EXAMPLES

The invention now being generally described, will be more readily understood by reference to the following examples, which are included merely for purposes of illustration of certain aspects and embodiments of the present invention, and are not intended to limit the invention.

### Example 1—Oral Efficacy of 6,8-Bis-benzylthio-octanoic Acid in Non-Small Cell Lung Cancer

Human H460 NSCLC cells were obtained from American Type Cell Culture (ATCC) (catalog no. HTB-177, Manassas, Va.). These cells tested negative for viral contamination using the Mouse Antibody Production (MAP) test, performed by Charles River Labs Molecular Division, upon the receipt of the tumor cells from ATCC. The tumor cells were maintained at 37° C. in a humidified 5% CO₂ atmosphere in T225 tissue culture flasks containing 50 mL of Roswell Park Memorial Institute (RPMI)-1640 solution with 10% Fetal Bovine Serum (FBS) and 2 mM L-glutamine. Cells were split at a ratio of 1:10 every 2-3 days by trypsinization and resuspended in fresh medium in a new flask. Cells were harvested for experiments in the same way at 70-90% confluency.

CD1-Nu/Nu female mice, ˜4-6 weeks old were obtained from Charles River Laboratories. Mice were housed 5 to a cage in a micro-isolator room in the Department of Animal Laboratory Research of New York State University (SUNY) at Stony Brook. Light-dark cycles were 12 h each daily, with light from 7 a.m. to 7 p.m. Food (Purina Rodent Chow) and water (distilled sterile-filtered water, pH 7) were provided ad libitum. Protocols and procedures were according to the rules of and approved by the SUNY Institutional Animal Care and Use Committee (IACUC).

An acclimation period of 7 days was allowed between the arrival of the animal at the study site before tumor inoculation and experimentation. Mice were inoculated subcutaneously (SC) in the right flank with 2×10⁶ human H460 NSCLC or BxPC3 pancreatic cancer cells that were suspended in 0.1 mL of Dulbeco's Phosphate Buffered Salt (PBS) solution using a 1 cc syringe with a 27⅝ gauge needle. Tumor dimensions (length and width) were measured daily before, during and after treatment (using Vernier calipers) and the tumor volume was calculated using the prolate ellipsoid formula: (length×width²)/2. Treatment with test or control articles began 8 days post tumor cell implantation when the tumor was approximately 300 mm³.

Oral dosing of 6,8-bis-benzylthio-octanoic acid was at 100 mg/kg with 11 animals per group. 100 mg of 6,8-bis-benzylthio-octanoic acid was suspended in a small volume 0.01-0.05N NaOH in 5% dextrose and titrated to pH 7.0 with 4% Glacial Acetic Acid to 50 mg/mL. Prior to administration the suspension was diluted with 5% dextrose to 12.5 mg/mL so that the animals received 100 mg/kg with a dose volume of about 0.2 mL delivered by gastric gavage. Post tumor cell implantation, mice were treated on day 8, day 15, day 22, and day 29.

A similar study was conducted in CD-1 nude mice (n=9) inoculated with 2×10⁶ BxPC-3 cells. The study was initiated when tumors reached an average size of 150 mm³ (day 0) and CPI-613 was administered at an oral dose of 100 mg/week for 4 weeks. A comparator arm (n=9) was conducted with IP treatment at a weekly dose of 25 mg/kg.

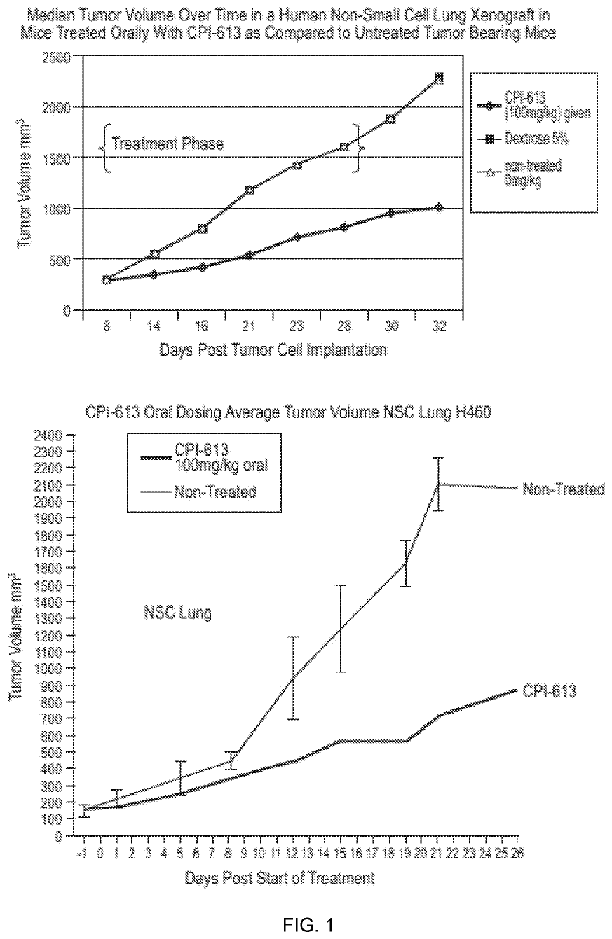

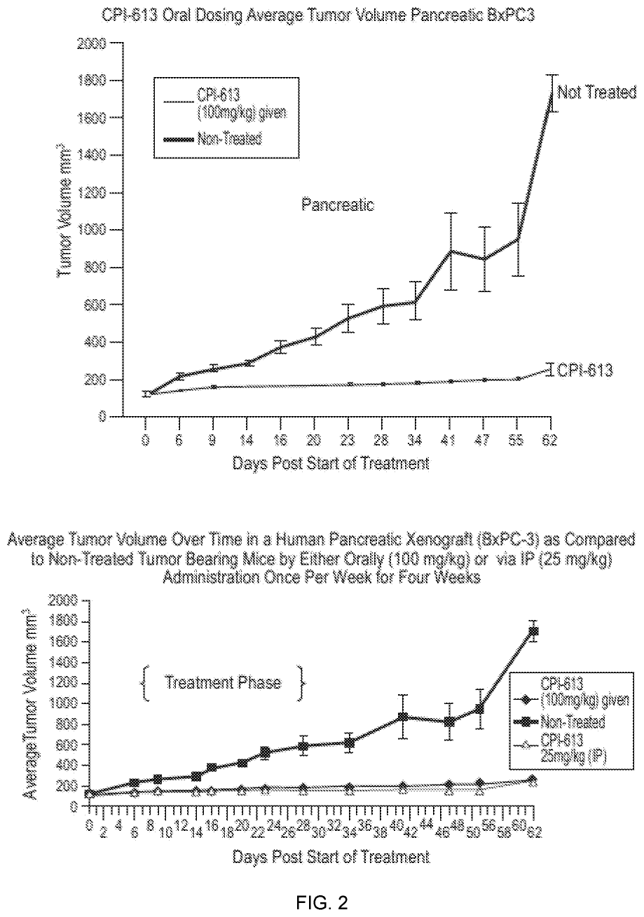

The results are presented in FIGS. 1 and 2. It is evident that the tumors in the mice treated with 6,8-bis-benzylthio-octanoic acid grew much more slowly than those in mice treated with 5% dextrose or untreated. The effect was especially pronounced in BxPC3 tumors. This example demonstrates that 6,8-bis-benzylthio-octanoic acid is effective to treat cancer when administered orally.

### Example 2—Oral Efficacy of 6,8-Bis-benzylthio-octanoic Acid in AML

C57Bl/6 mice were injected into their tail veins with 1 million MFL2 cells (Pardee, T. S. et al., Experimental Hematology, 2011, 39, 473-485) on Day 0 and beginning on Day 7, upon confirmation of engraftment by bioluminescence imaging, were treated by gavage with CPI-613 (300 mg/kg daily except weekends of a 50 mg/mL solution in 0.05 N NaOH in 5% dextrose, adjusted to pH 7.5-8 with 4% glacial acetic acid; 1 animal for the chloroquine experiment), intraperitoneally (IP) with chloroquine (200 μL (ca. 100 mg/kg) daily except weekends of a 10 mg/mL solution in PBS; Chlr; 3 animals), orally with metformin (1 mg/mL in the drinking water with ad lib access), or with a combination of CPI-613 (300 mg/kg daily gavage as above) and either chloroquine (200 μL daily IP as above; 4 animals) or metformin (1 mg/mL ad lib in drinking water as above) and followed for survival. Control animals (1 animal in chloroquine experiment) received both oral and IP vehicles. P value was determined by log rank test.

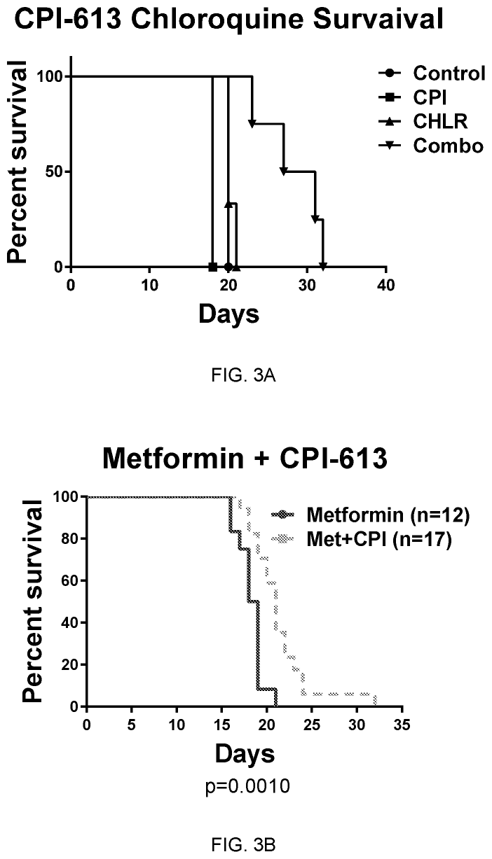

The results are presented in FIGS. 3A and 3B. This example demonstrates that oral 6,8-bis-benzylthio-octanoic acid significantly prolongs survival of AML tumor-bearing mice, especially when combined with either chloroquine or metformin.

### Example 3—Oral Efficacy of 6,8-Bis-benzylthio-octanoic Acid in ALL

Balb/c mice were injected into their tail veins with 1 million Baf3-p210 cells on Day 0 and beginning on Day 3, upon confirmation of engraftment by bioluminescence imaging, were treated with saline (control), doxorubicin (3 mg/kg IP in 200 μL PBS), or doxorubicin (3 mg/kg IP in 200 μL PBS) plus CPI-613 (250 mg/kg gavage of a 25 mg/mL solution of CPI-613 in 0.05 N NaOH in 5% dextrose, adjusted to pH 7.5-8 with 4% glacial acetic acid). and followed for survival. Doxorubicin was administered once daily for three consecutive days, and CPI-613 was administered once daily until death. P value was determined by log rank test.

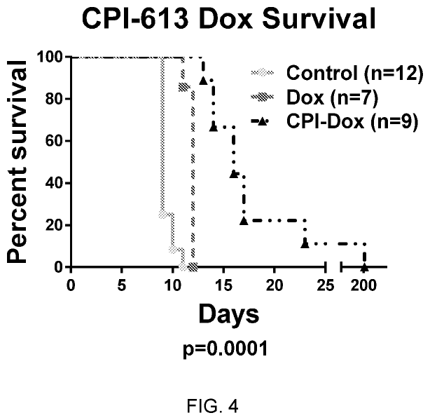

The results are presented in FIG. 4. This example demonstrates that oral 6,8-bis-benzylthio-octanoic acid combined with doxorubicin significantly prolongs survival of Philadelphia chromosome positive B cell ALL tumor-bearing mice compared to doxorubicin alone.

### Example 4—Salts of 6,8-Bis-benzylthio-octanoic Acid

### General Procedures

Differential scanning calorimetry was performed using TA Instruments Q2000. Temperature calibration was performed using NIST traceable indium metal. The sample was placed into an aluminum crimped pan with manual pinhole, and the weight was accurately recorded. A weighed aluminum pan configured as the sample pan was placed on the reference side of the cell. The sample was heated using a heating rate of 10° C./min from −50° C. to 250° C.

Solution proton nuclear magnetic resonance spectra were acquired at 25° C. with an Agilent DD2-400 spectrometer at a ¹H Larmor frequency of 399.82 MHz. The sample was dissolved in DMSO-d₆ containing tetramethylsilane (TMS). The spectrum was acquired with a ¹H pulse width of 6.5 μs, a 5 second acquisition time, a 2.5 second delay between scans, a spectral width of 6410 Hz with 64102 data points, and 40 co-added scans. The free induction decay (FID) was processed using Agilent VnmrJ 3.2A software with 131072 points and an exponential line broadening factor of 0.2 Hz to improve the signal-to-noise ratio. The residual peak from incompletely deuterated solvent is at approximately 2.50 ppm. The spectrum was referenced to internal tetramethylsilane (TMS) at 0.0 ppm.

X-ray powder diffraction patterns were collected using a PANalytical X'Pert PRO MPD diffractometer. The specimen was analyzed using Cu radiation produced using an Optix long fine-focus source. An elliptically graded multilayer mirror was used to focus the Cu KαX-rays of the source through the specimen and onto the detector. The specimen was sandwiched between 3-micron thick films, analyzed in transmission geometry, and rotated parallel to the diffraction vector to optimize orientation statistics. A beam-stop, short antiscatter extension, antiscatter knife edge, and helium purge were used to minimize the background generated by air scattering. Soller slits were used for the incident and diffracted beams to minimize axial divergence. Diffraction patterns were collected using a scanning position-sensitive detector (X'Celerator) located 240 mm from the specimen. Prior to the analysis a silicon specimen (NIST standard reference material 640d) was analyzed to verify the position of the silicon 111 peak. Figures of x-ray powder diffraction patterns were generated using unvalidated software PatternMatch v3.0.4 and are non-cGMP representations.

Infrared spectra were acquired on a Nexus 670® Fourier transform infrared (FT-IR) spectrophotometer (Thermo Nicolet) equipped with an Ever-Glo mid/far IR source, a potassium bromide (KBr) beamsplitter, and a deuterated triglycine sulfate (DTGS) detector. Wavelength verification was performed using NIST SRM 1921b (polystyrene). An attenuated total reflectance (ATR) accessory (Thunderdome™, Thermo Spectra-Tech), with a germanium (Ge) crystal was used for data acquisition. Each spectrum represents 256 co-added scans collected at a spectral resolution of 4 cm⁻¹. A background data set was acquired with a clean Ge crystal. A Log 1/R (R=reflectance) spectrum was obtained by taking a ratio of these two data sets against each other.

Thermogravimetric analysis was performed using a TA Instruments Model 2050. Temperature calibration was performed using nickel and Alumel™. Each sample was placed in an aluminum pan. The sample was hermetically sealed, the lid pierced, then inserted into the thermogravimetric furnace. The furnace was heated under nitrogen. The sample was heated using a heating rate of 10° C./min from ambient to 250° C., 300° C., or 350° C.

### Piperazine

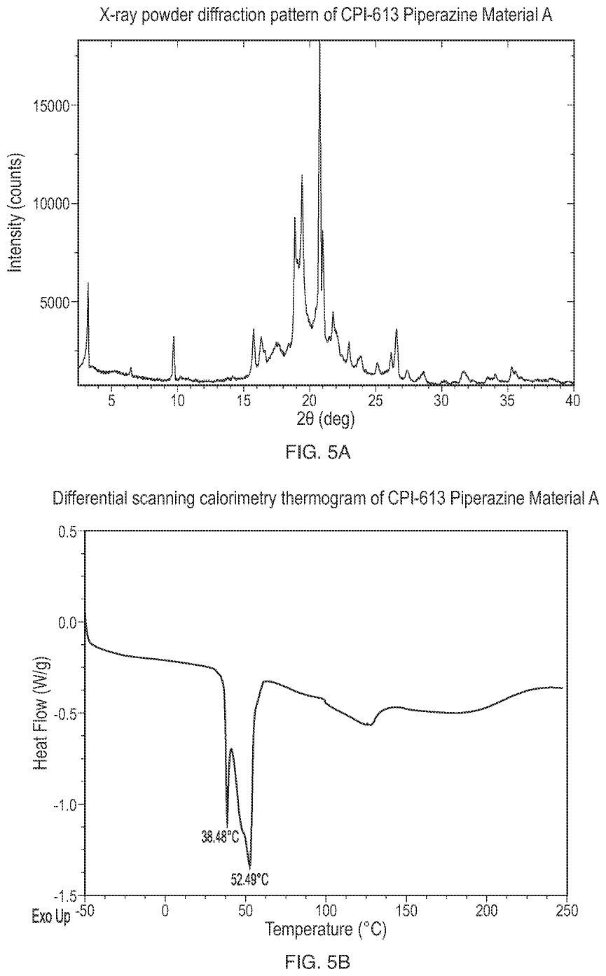

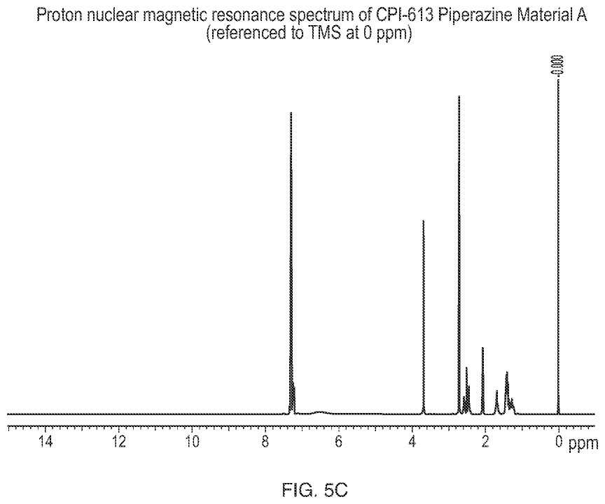

6,8-bis-benzylthio-octanoic acid (66.8 mg) was charged to a glass vial and contacted with an ether solution containing approximately one molar equivalent of piperazine (14.8 mg in 1.1 mL) resulting in the dissolution followed by oil formation. The sample was refrigerated (2 to 8° C.) overnight resulting in nucleation. The sample was transferred to a freezer for approximately 3 days. The supernatant was decanted and solids briefly dried under nitrogen to provide a piperazine salt of 6,8-bis-benzylthio-octanoic acid as a crystalline, anhydrous material with a 2:1 stoichiometry of 6,8-bis-benzylthio-octanoic acid to piperazine, denoted CPI-613 piperazine material A. Differential scanning calorimetry shows endothermic events with an onset at 38° C. FIGS. 5A, 5B, and 5C present an X-ray powder diffraction pattern, differential scanning calorimetry thermogram, and proton nuclear magnetic resonance spectrum of CPI-613 piperazine material A. Peaks observed in the x-ray powder diffraction pattern of CPI-613 piperazine material A are listed in the table below.

| °2θ (±0.20) | d space (Å) | Intensity (%) |
|---|---|---|
| 3.22 | 27.376 ± 1.697 | 32 |
| 6.47 | 13.642 ± 0.421 | 9 |
| 9.72 | 9.092 ± 0.187 | 18 |
| 15.76 | 5.620 ± 0.071 | 20 |
| 16.34 | 5.421 ± 0.066 | 18 |
| 18.89 | 4.695 ± 0.049 | 51 |
| 19.43 | 4.566 ± 0.047 | 63 |
| 20.75 | 4.277 ± 0.041 | 100 |
| 21.00 | 4.226 ± 0.040 | 47 |
| 21.76 | 4.081 ± 0.037 | 25 |
| 22.96 | 3.870 ± 0.033 | 16 |
| 23.83 | 3.731 ± 0.031 | 12 |
| 25.12 | 3.542 ± 0.028 | 11 |
| 26.16 | 3.403 ± 0.026 | 13 |
| 26.56 | 3.354 ± 0.025 | 20 |

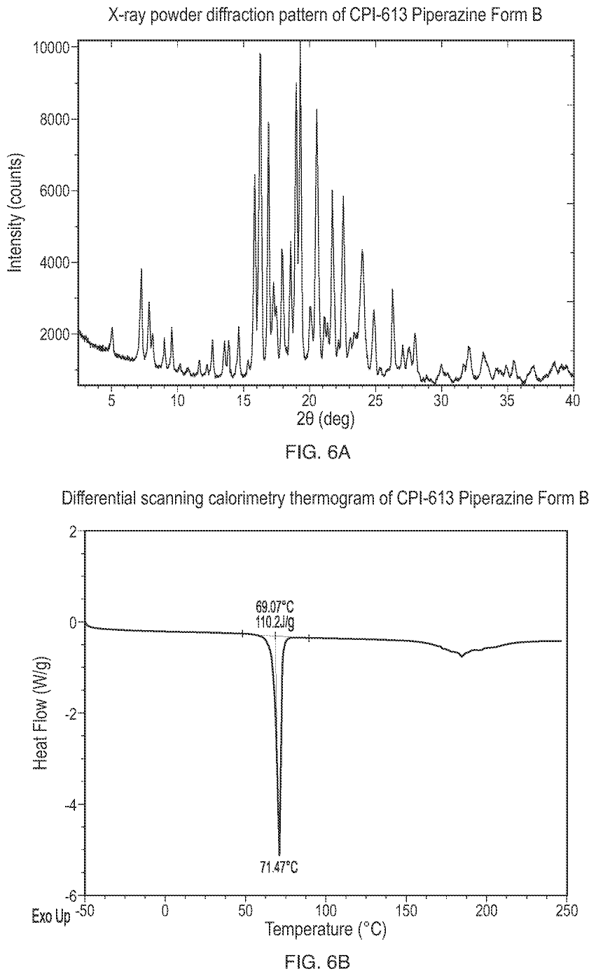

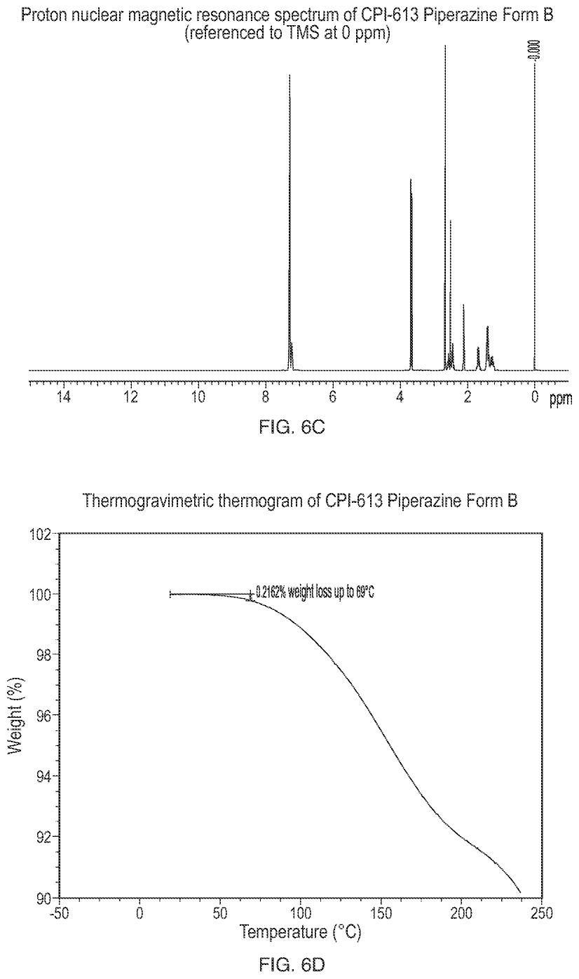

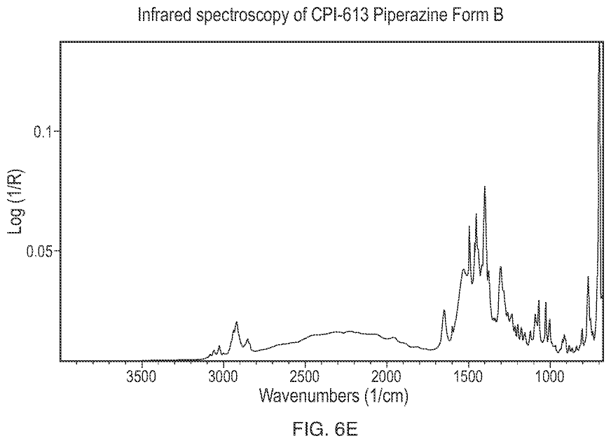

6,8-bis-benzylthio-octanoic acid (105.2 mg) and diethyl ether (2.0 mL) were charged to a glass vial. The mixture was sonicated, generating a clear solution. The solution was seeded with a small quantity of CPI-613 piperazine material A. The sample was placed onto a stir plate and stirred at approximately 300 RPM. An ether solution containing approximately ½ molar equivalent of piperazine (12.5 mg in 2 mL) was added to the solution resulting in precipitation. After approximately 5 minutes, additional diethyl ether (3 mL) was added to the slurry along with additional seeds of CPI-613 piperazine material A, and then followed by sonication. The slurry was returned to stir plate and treated with additional ether (9 mL). After approximately 4 days the solids were harvested by vacuum filtration and briefly dried under nitrogen to provide 96.5 mg of a crystalline, anhydrous material with a 2:1 stoichiometry of 6,8-bis-benzylthio-octanoic acid to piperazine, denoted CPI-613 piperazine form B. Differential scanning calorimetry shows an endothermic event with an onset at 69° C. concurrent with negligible weight loss by thermogravimetric analysis (0.2% up to 69° C.). FIGS. 6A, 6B, 6C, 6D, and 6E present an X-ray powder diffraction pattern, differential scanning calorimetry thermogram, proton nuclear magnetic resonance spectrum, thermogravimetric thermogram, and infrared spectroscopy of CPI-613 piperazine form B. Peaks observed in the infrared spectrum of CPI-613 piperazine form B include 698, 701, 754, 767, 804, 840, 864, 884, 913, 924, 1003, 1027, 1070, 1092, 1122, 1155, 1177, 1199, 1216, 1234, 1260, 1303, 1338, 1378, 1400, 1453, 1462, 1494, 1530, 1600, 1649, 2854, 2922, 3027, and 3060 (all ±4 cm⁻¹). Peaks observed in the x-ray powder diffraction pattern of CPI-613 piperazine form B are listed in the table below.

| °2θ (±0.20) | d space (Å) | Intensity (%) |
|---|---|---|
| 5.09 | 17.343 ± 0.681 | 21 |
| 7.30 | 12.099 ± 0.331 | 37 |
| 7.90 | 11.185 ± 0.283 | 28 |
| 8.16 | 10.827 ± 0.265 | 19 |
| 9.04 | 9.774 ± 0.216 | 18 |
| 9.62 | 9.184 ± 0.190 | 21 |
| 10.23 | 8.639 ± 0.168 | 11 |
| 10.83 | 8.161 ± 0.150 | 10 |
| 11.70 | 7.556 ± 0.129 | 12 |
| 12.27 | 7.208 ± 0.117 | 11 |
| 12.69 | 6.970 ± 0.109 | 18 |
| 13.61 | 6.503 ± 0.095 | 18 |
| 13.92 | 6.355 ± 0.091 | 17 |
| 14.68 | 6.031 ± 0.082 | 21 |
| 15.38 | 5.755 ± 0.074 | 12 |
| 15.88 | 5.575 ± 0.070 | 63 |
| 16.31 | 5.430 ± 0.066 | 96 |
| 16.92 | 5.235 ± 0.061 | 77 |
| 17.31 | 5.118 ± 0.059 | 33 |
| 17.51 | 5.060 ± 0.057 | 27 |
| 17.98 | 4.930 ± 0.054 | 42 |
| 18.62 | 4.762 ± 0.051 | 45 |
| 19.03 | 4.660 ± 0.049 | 87 |
| 19.35 | 4.584 ± 0.047 | 100 |
| 20.12 | 4.410 ± 0.043 | 27 |
| 20.60 | 4.309 ± 0.041 | 81 |
| 21.16 | 4.195 ± 0.039 | 24 |
| 21.40 | 4.150 ± 0.038 | 22 |
| 21.78 | 4.078 ± 0.037 | 59 |
| 22.24 | 3.994 ± 0.035 | 18 |
| 22.59 | 3.932 ± 0.034 | 57 |
| 23.12 | 3.844 ± 0.033 | 18 |
| 24.07 | 3.695 ± 0.030 | 41 |
| 24.92 | 3.570 ± 0.028 | 26 |
| 25.38 | 3.506 ± 0.027 | 10 |
| 26.35 | 3.379 ± 0.025 | 32 |
| 27.12 | 3.286 ± 0.024 | 16 |
| 27.60 | 3.229 ± 0.023 | 16 |
| 28.02 | 3.182 ± 0.022 | 20 |

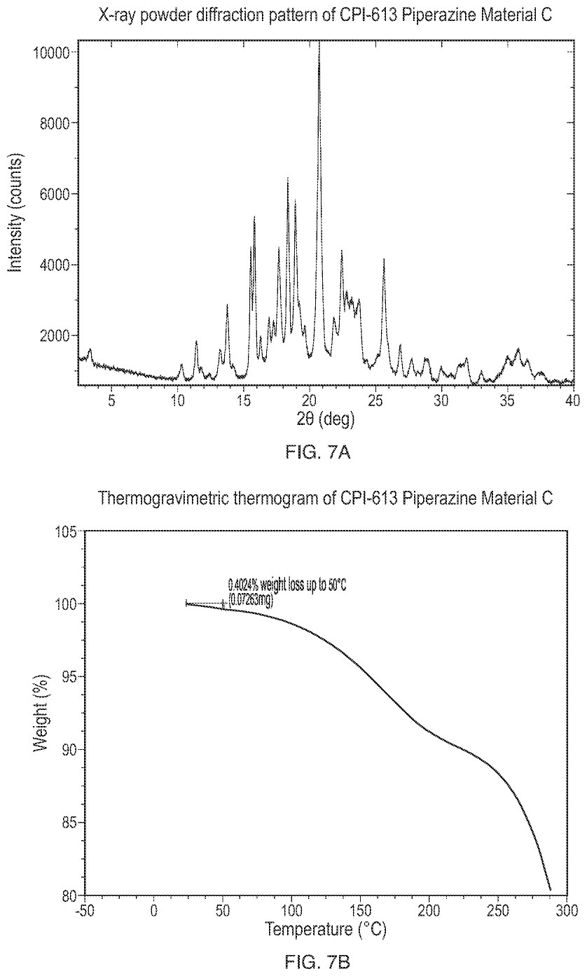

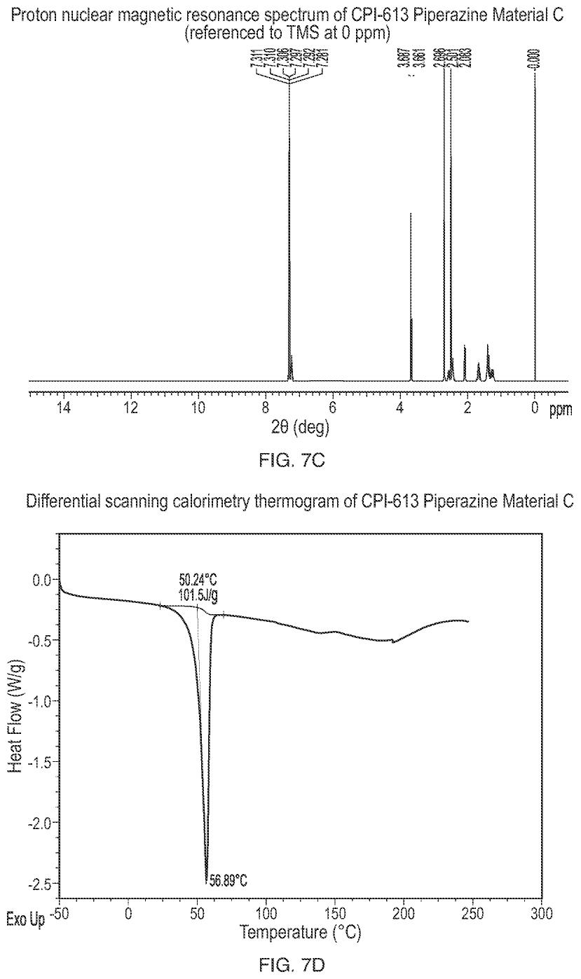

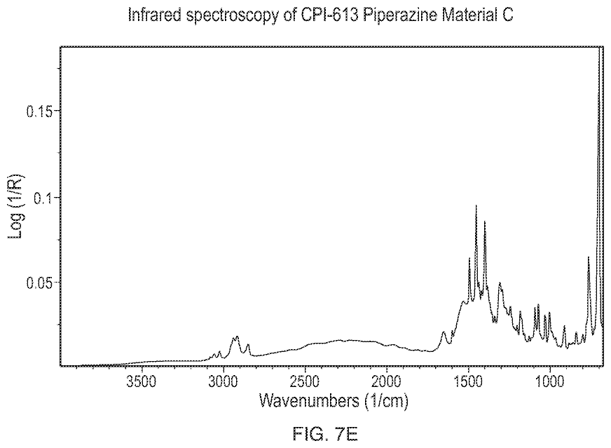

6,8-bis-benzylthio-octanoic acid (120.2 mg) was charged to a glass vial. A solution containing approximately a molar equivalent of piperazine (26.5 mg in 5 mL of diethyl ether) was added resulting in the oiling. An additional 13 mL of diethyl ether was added with sonication prior to refrigerated storage. After overnight storage the sample was harvested by vacuum filtration. The gel-like, thick filter cake collapsed to white, opaque solids when briefly dried under nitrogen to provide a crystalline material with a 2:1 stoichiometry of 6,8-bis-benzylthio-octanoic acid to piperazine, denoted CPI-613 piperazine material C. Differential scanning calorimetry shows an endothermic event with an onset at 50° C. concurrent with a 0.4% weight loss up to 50° C. by thermogravimetric analysis. Crystallization solvent was not observed in the solution proton nuclear magnetic resonance spectrum. FIGS. 7A, 7B, 7C, 7D, and 7E present an X-ray powder diffraction pattern, thermogravimetric thermogram, proton nuclear magnetic resonance spectrum, differential scanning calorimetry thermogram, and infrared spectroscopy of CPI-613 piperazine material C. Peaks observed in the infrared spectrum of CPI-613 piperazine material C include 701, 763, 799, 839, 912, 1003, 1030, 1071, 1092, 1128, 1182, 1201, 1242, 1307, 1340, 1384, 1421, 1437, 1453, 1494, 1532, 1599, 1652, 2851, 2918, 2940, 3025, and 3059 (all ±4 cm⁻¹). Peaks observed in the x-ray powder diffraction pattern of CPI-613 piperazine material C are listed in the table below.

| °2θ (±0.20) | d space (Å) | Intensity (%) |
|---|---|---|
| 3.39 | 26.051 ± 1.537 | 16 |
| 10.30 | 8.578 ± 0.166 | 11 |
| 11.43 | 7.738 ± 0.135 | 18 |
| 11.81 | 7.485 ± 0.126 | 11 |
| 13.24 | 6.682 ± 0.100 | 15 |
| 13.78 | 6.422 ± 0.093 | 27 |
| 15.56 | 5.692 ± 0.073 | 43 |
| 15.83 | 5.592 ± 0.070 | 51 |
| 16.30 | 5.435 ± 0.066 | 19 |
| 16.93 | 5.234 ± 0.061 | 24 |
| 17.27 | 5.130 ± 0.059 | 23 |
| 17.67 | 5.014 ± 0.056 | 43 |
| 18.36 | 4.828 ± 0.052 | 62 |
| 18.93 | 4.685 ± 0.049 | 56 |
| 19.64 | 4.516 ± 0.046 | 22 |
| 20.73 | 4.282 ± 0.041 | 100 |
| 21.86 | 4.063 ± 0.037 | 24 |
| 22.44 | 3.959 ± 0.035 | 42 |
| 22.79 | 3.899 ± 0.034 | 31 |
| 23.21 | 3.830 ± 0.033 | 30 |
| 23.74 | 3.745 ± 0.031 | 29 |
| 25.62 | 3.474 ± 0.027 | 40 |
| 26.85 | 3.318 ± 0.024 | 16 |
| 27.72 | 3.216 ± 0.023 | 13 |

### Benzathine

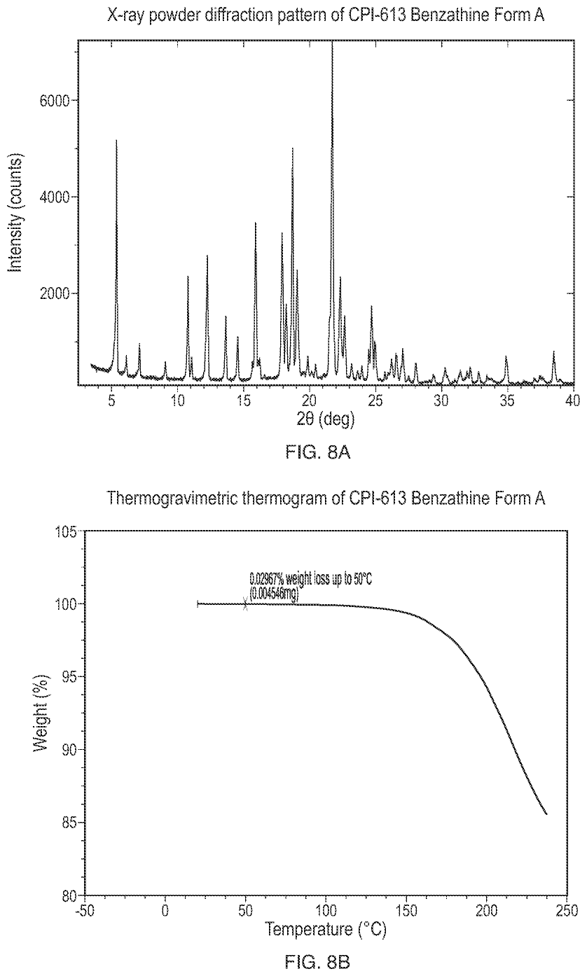

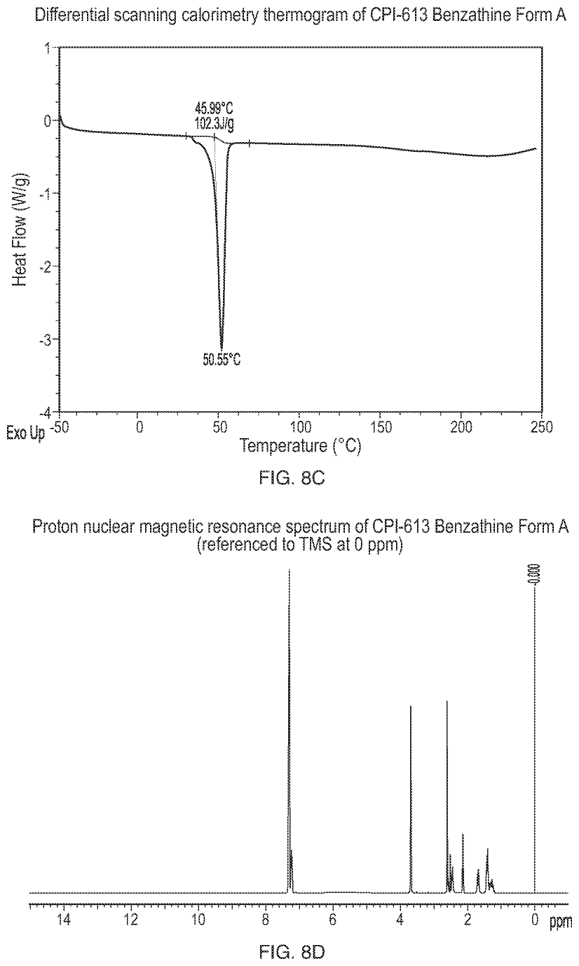

6,8-bis-benzylthio-octanoic acid (41.6 mg) was charged to a glass vial. A molar equivalent of benzathine (25.1 μL in 2 mL of isopropyl alcohol) was added to the vial resulting in a clear solution. The solution was contacted with 2 mL of ethyl acetate and refrigerated (2 to 8° C.) overnight. The clear solution was then transferred to a freezer (−25 to −10° C.) for approximately 3 days, no solids were observed. The solution was evaporated under nitrogen, generating an oil. The oil was contacted with 5 mL of isopropyl ether and sonicated prior to storage in the freezer (−25 to −10° C.) for 9 days. Upon removal, the solution was decanted and the remaining oil placed briefly under nitrogen. The sample was then exposed to vacuum at ambient temperature overnight resulting in a crystalline, anhydrous material with a 2:1 stoichiometry of 6,8-bis-benzylthio-octanoic acid to benzathine, denoted CPI-613 benzathine form A. Differential scanning calorimetry shows an endothermic event with an onset at 46° C. concurrent with negligible weight loss (<0.03% up to 50° C.) by thermogravimetric analysis. The tentative unit cell parameters and calculated volume of CPI-613 benzathine form A at ambient temperature, derived from indexing, are: a=5.773 Å, b=15.105 Å, c=16.902 Å, α=103.11°, β=92.62°, γ=142.00°, V=1406.0 Å³. The space group may either be P1 (1) or P-1 (2). FIGS. 8A, 8B, 8C, and 8D present an X-ray powder diffraction pattern, differential scanning calorimetry thermogram, proton nuclear magnetic resonance spectrum, and thermogravimetric thermogram of CPI-613 benzathine form A. Peaks observed in the x-ray powder diffraction pattern of CPI-613 benzathine form A are listed in the table below.

| °2θ (±0.20) | d space (Å) | Intensity (%) |
|---|---|---|
| 5.43 | 16.269 ± 0.599 | 70 |
| 6.16 | 14.343 ± 0.465 | 10 |
| 7.16 | 12.336 ± 0.344 | 13 |
| 9.12 | 9.688 ± 0.212 | 8 |
| 10.83 | 8.163 ± 0.150 | 32 |
| 11.10 | 7.961 ± 0.143 | 9 |
| 12.30 | 7.190 ± 0.116 | 38 |
| 13.68 | 6.468 ± 0.094 | 20 |
| 14.58 | 6.069 ± 0.083 | 15 |
| 15.71 | 5.638 ± 0.071 | 8 |
| 15.95 | 5.552 ± 0.069 | 48 |
| 16.25 | 5.449 ± 0.067 | 9 |
| 17.94 | 4.941 ± 0.055 | 44 |
| 18.27 | 4.852 ± 0.053 | 24 |
| 18.73 | 4.733 ± 0.050 | 69 |
| 19.08 | 4.648 ± 0.048 | 34 |
| 19.89 | 4.460 ± 0.044 | 10 |
| 20.19 | 4.394 ± 0.043 | 5 |
| 20.49 | 4.331 ± 0.042 | 7 |
| 21.73 | 4.087 ± 0.037 | 100 |
| 22.35 | 3.975 ± 0.035 | 31 |
| 22.68 | 3.917 ± 0.034 | 21 |
| 23.21 | 3.829 ± 0.033 | 7 |
| 23.67 | 3.756 ± 0.031 | 5 |
| 24.00 | 3.706 ± 0.030 | 7 |
| 24.52 | 3.628 ± 0.029 | 11 |
| 24.72 | 3.598 ± 0.029 | 24 |
| 24.99 | 3.560 ± 0.028 | 13 |
| 25.72 | 3.460 ± 0.026 | 5 |
| 26.23 | 3.395 ± 0.025 | 9 |
| 26.60 | 3.349 ± 0.025 | 9 |
| 27.09 | 3.289 ± 0.024 | 11 |
| 28.06 | 3.177 ± 0.022 | 8 |

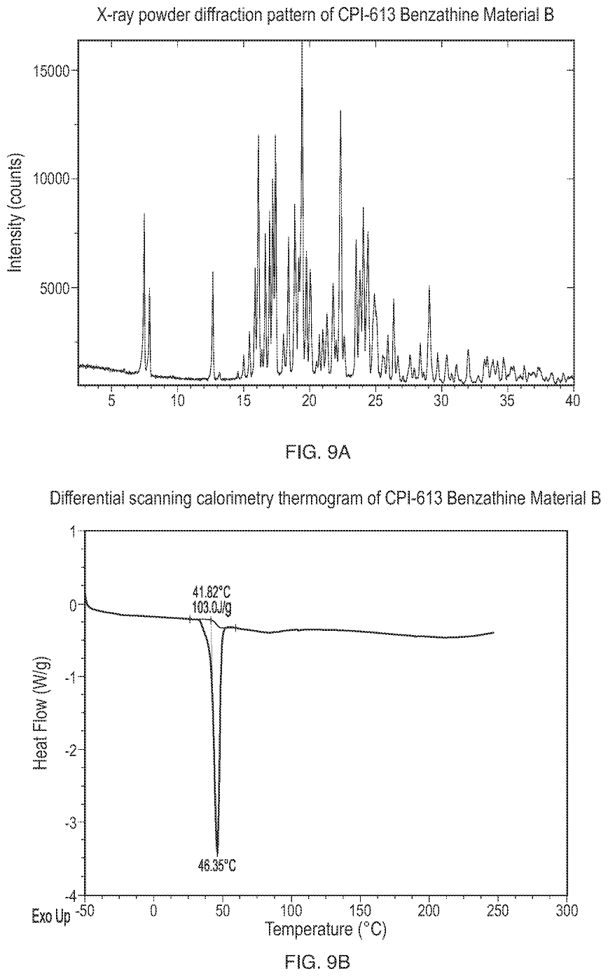

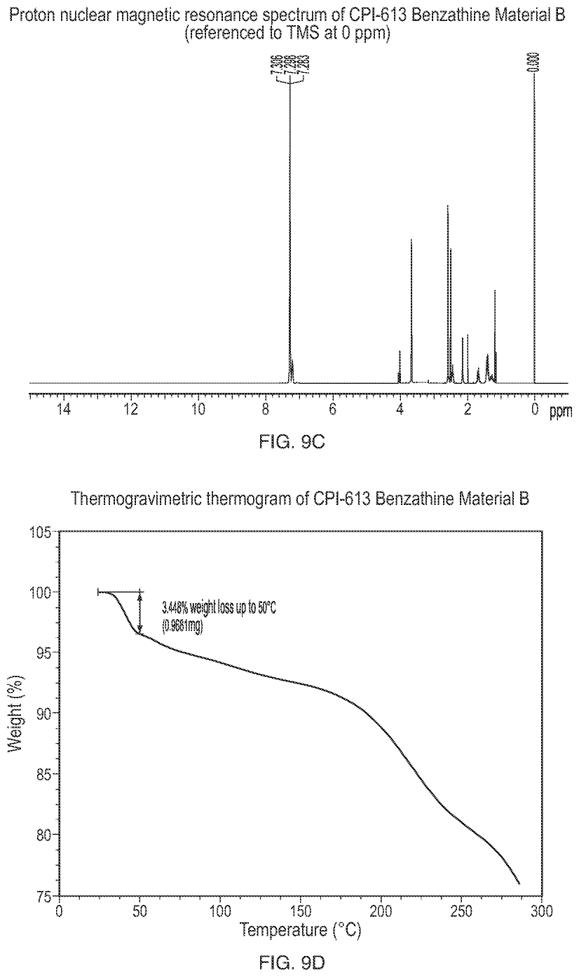

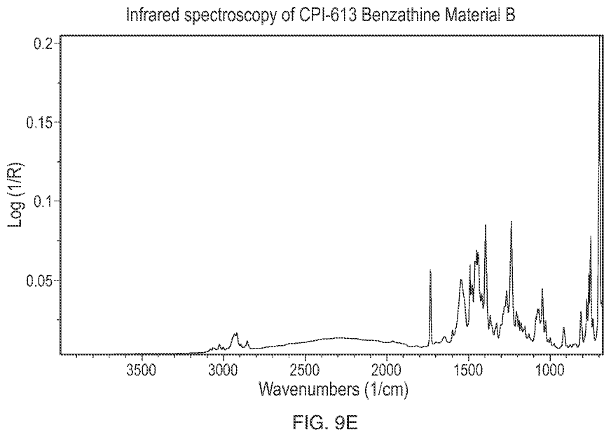

6,8-bis-benzylthio-octanoic acid (123.9 mg) and 0.5 mL of ethyl acetate were charged to a glass vial resulting in a clear solution. A ½ molar equivalent of benzathine (37 μL) was added to the sample. The clear solution was transferred to a freezer (−25 to −10° C.) overnight, resulting in the formation of fine birefringent aciculars. An additional 0.5 mL of ethyl acetate was added to the sample followed by freezer (−25 to −10° C.) storage overnight. Solids were harvested by vacuum filtration resulting in a crystalline material with a 2:1 stoichiometry of 6,8-bis-benzylthio-octanoic acid to benzathine, denoted CPI-613 benzathine material B. Differential scanning calorimetry shows an endothermic event with an onset at 42° C. CPI-613 benzathine material B exhibits 3.4% weight loss up to 50° C. by thermogravimetric analysis. Based on peak integrations, the solution proton nuclear magnetic resonance spectrum indicates the material contains 0.3 moles of ethyl acetate per mole of 6,8-bis-benzylthio-octanoic acid (providing a stoichiometric ratio of 2:1:0.6 for 6,8-bis-benzylthio-octanoic acid/benzathine/ethyl acetate). The quantity of ethyl acetate determine by NMR is consistent with the weight loss observed by thermogravimetric analysis. However, it is unknown whether ethyl acetate is residual or if CPI-613 benzathine material B is an ethyl acetate solvate. FIGS. 9A, 9B, 9C, 9D, and 9E present an X-ray powder diffraction pattern, differential scanning calorimetry thermogram, proton nuclear magnetic resonance spectrum, thermogravimetric thermogram, and infrared spectroscopy of CPI-613 benzathine material B. Peaks observed in the infrared spectrum of CPI-613 benzathine material B include 687, 699, 740, 754, 766, 778, 813, 845, 880, 920, 1001, 1015, 1031, 1050, 1073, 1080, 1090, 1133, 1158, 1181, 1194, 1210, 1240, 1269, 1331, 1369, 1398, 1421, 1443, 1452, 1463, 1480, 1494, 1547, 1600, 1650, 1736, 2856, 2920, 3029, and 3065 (all ±4 cm⁻¹). Peaks observed in the x-ray powder diffraction pattern of CPI-613 benzathine material B are listed in the table below.

| °2θ (±0.20) | d space (Å) | Intensity (%) |
|---|---|---|
| 7.48 | 11.802 ± 0.315 | 50 |
| 7.91 | 11.173 ± 0.282 | 31 |
| 12.69 | 6.972 ± 0.109 | 35 |
| 13.19 | 6.706 ± 0.101 | 7 |
| 14.58 | 6.069 ± 0.083 | 7 |
| 15.02 | 5.892 ± 0.078 | 12 |
| 15.47 | 5.725 ± 0.074 | 18 |
| 15.88 | 5.578 ± 0.070 | 36 |
| 16.14 | 5.486 ± 0.068 | 74 |
| 16.39 | 5.405 ± 0.066 | 13 |
| 16.66 | 5.319 ± 0.063 | 45 |
| 16.99 | 5.215 ± 0.061 | 52 |
| 17.23 | 5.144 ± 0.059 | 61 |
| 17.43 | 5.083 ± 0.058 | 73 |
| 18.04 | 4.913 ± 0.054 | 18 |
| 18.41 | 4.815 ± 0.052 | 45 |
| 18.90 | 4.693 ± 0.049 | 54 |
| 19.19 | 4.622 ± 0.048 | 39 |
| 19.45 | 4.560 ± 0.046 | 100 |
| 19.76 | 4.489 ± 0.045 | 41 |
| 20.07 | 4.421 ± 0.044 | 36 |
| 20.53 | 4.323 ± 0.042 | 10 |
| 20.74 | 4.279 ± 0.041 | 17 |
| 21.01 | 4.225 ± 0.040 | 19 |
| 21.33 | 4.163 ± 0.039 | 24 |
| 21.78 | 4.077 ± 0.037 | 31 |
| 22.02 | 4.034 ± 0.036 | 16 |
| 22.34 | 3.977 ± 0.035 | 79 |
| 22.63 | 3.926 ± 0.034 | 17 |
| 23.53 | 3.778 ± 0.032 | 44 |
| 23.82 | 3.733 ± 0.031 | 35 |
| 24.08 | 3.693 ± 0.030 | 53 |
| 24.41 | 3.643 ± 0.029 | 46 |
| 24.92 | 3.570 ± 0.028 | 29 |
| 25.07 | 3.550 ± 0.028 | 24 |
| 25.54 | 3.485 ± 0.027 | 12 |
| 25.64 | 3.471 ± 0.027 | 11 |
| 25.93 | 3.434 ± 0.026 | 17 |
| 26.38 | 3.376 ± 0.025 | 27 |
| 26.69 | 3.338 ± 0.025 | 12 |
| 27.07 | 3.292 ± 0.024 | 6 |
| 27.60 | 3.229 ± 0.023 | 12 |
| 27.93 | 3.192 ± 0.022 | 8 |
| 28.37 | 3.143 ± 0.022 | 15 |
| 29.06 | 3.070 ± 0.021 | 31 |
| 29.70 | 3.006 ± 0.020 | 12 |

### Lysine

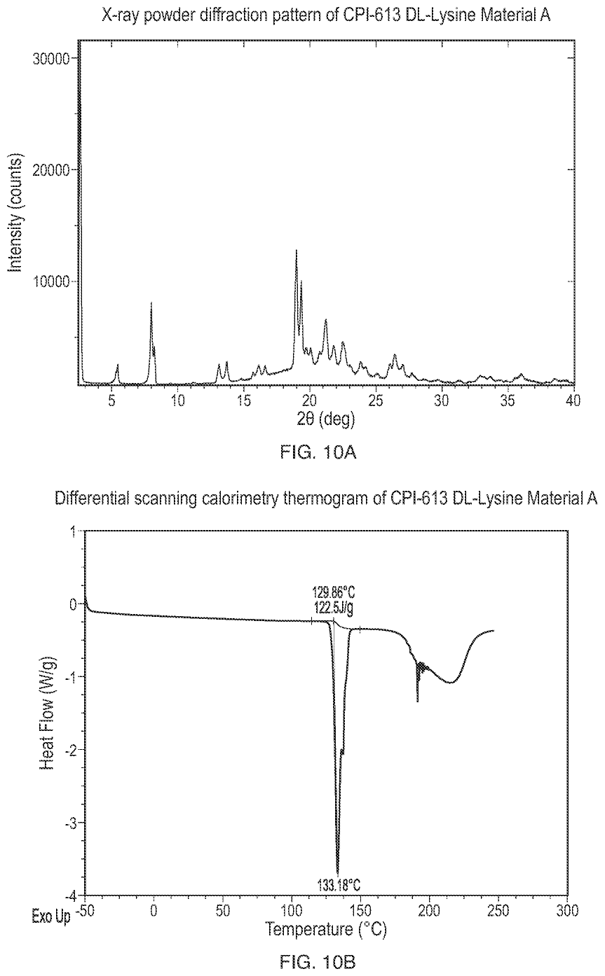

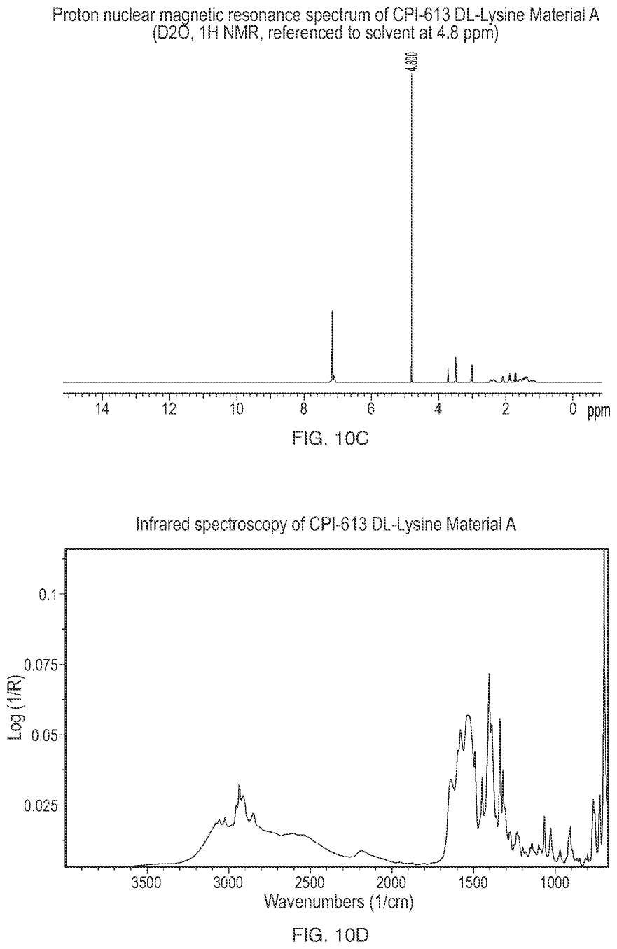

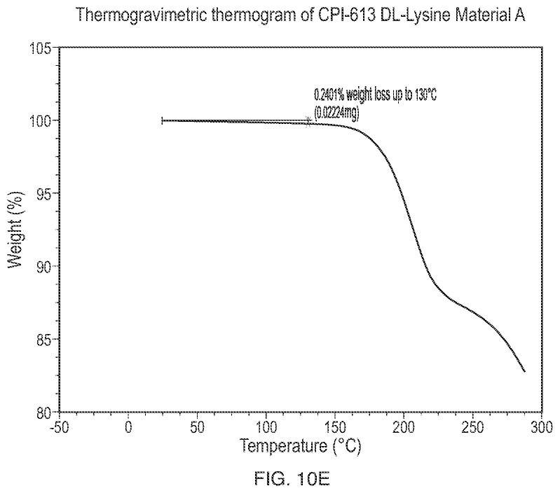

6,8-bis-benzylthio-octanoic acid (106.6 mg) and approximately a molar equivalent of DL-lysine (44.7 mg) were charged to a glass vial. The sample was then contacted with 3 mL of methanol resulting in a hazy solution. An additional 1 mL of methanol was added and the sample heated to 50° C. resulting in a hazy solution. The sample was removed and refrigerated (2 to 8° C.) for approximately 3 days. The sample was observed to contain limited fines and was transferred to a freezer (−25 to −10° C.) for approximately 21 days. The solids were harvested by decanting the solution and drying the resulting solids under nitrogen. The solids were observed to flow when pressed. The sample was stored in the freezer after isolation/prior to analysis resulting in a crystalline, anhydrous salt with a 1:1 stoichiometry of 6,8-bis-benzylthio-octanoic acid to DL-lysine, denoted CPI-613 DL-lysine material A. Differential scanning calorimetry shows an endothermic event with an onset at 130° C. and exhibits negligible weight loss (0.2%) up to 130° C. by thermogravimetric analysis. FIGS. 10A, 10B, 10C, 10D, and 10E present an X-ray powder diffraction pattern, differential scanning calorimetry thermogram, proton nuclear magnetic resonance spectrum, infrared spectroscopy, and thermogravimetric thermogram of CPI-613 DL-lysine material A. Peaks observed in the infrared spectrum of CPI-613 DL-lysine material A include 699, 730, 761, 769, 804, 852, 910, 974, 1029, 1069, 1104, 1145, 1184, 1200, 1237, 1275, 1323, 1341, 1391, 1407, 1450, 1495, 1543, 1582, 1643, 2189, 2851, 2913, 2936, 2954, 3024, and 3060 (all ±4 cm⁻¹). Peaks observed in the x-ray powder diffraction pattern of CPI-613 DL-lysine material A are listed in the table below.

| °2θ (±0.20) | d space (Å) | Intensity (%) |
|---|---|---|
| 2.67 | 33.009 ± 2.468 | 100 |
| 5.50 | 16.052 ± 0.583 | 8 |
| 8.05 | 10.972 ± 0.272 | 26 |
| 8.27 | 10.684 ± 0.258 | 13 |
| 13.15 | 6.727 ± 0.102 | 8 |
| 13.73 | 6.447 ± 0.093 | 9 |
| 15.73 | 5.631 ± 0.071 | 6 |
| 16.13 | 5.491 ± 0.068 | 8 |
| 16.62 | 5.331 ± 0.064 | 8 |
| 18.98 | 4.673 ± 0.049 | 41 |
| 19.34 | 4.585 ± 0.047 | 32 |
| 19.74 | 4.494 ± 0.045 | 13 |
| 20.06 | 4.422 ± 0.044 | 13 |
| 21.19 | 4.189 ± 0.039 | 21 |
| 21.80 | 4.074 ± 0.037 | 13 |
| 22.50 | 3.949 ± 0.035 | 15 |
| 23.82 | 3.733 ± 0.031 | 9 |
| 24.17 | 3.679 ± 0.030 | 7 |
| 26.03 | 3.420 ± 0.026 | 8 |
| 26.41 | 3.372 ± 0.025 | 11 |
| 27.00 | 3.300 ± 0.024 | 8 |

### Triethanolamine

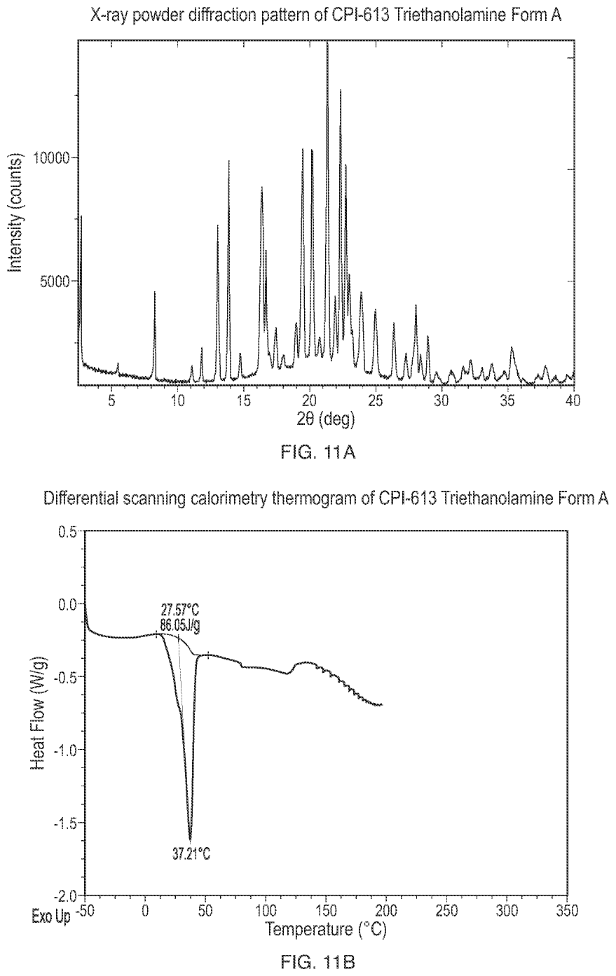

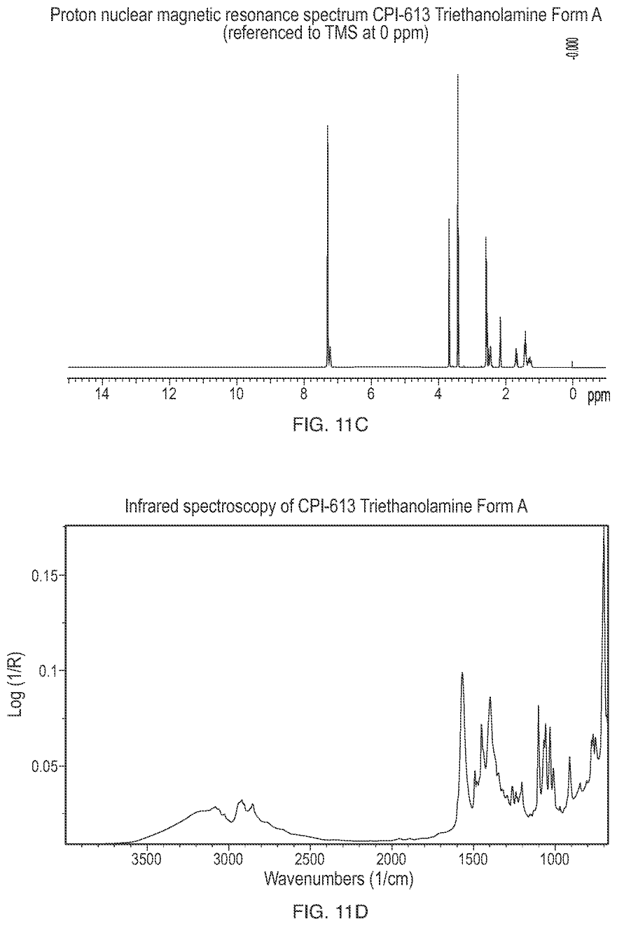

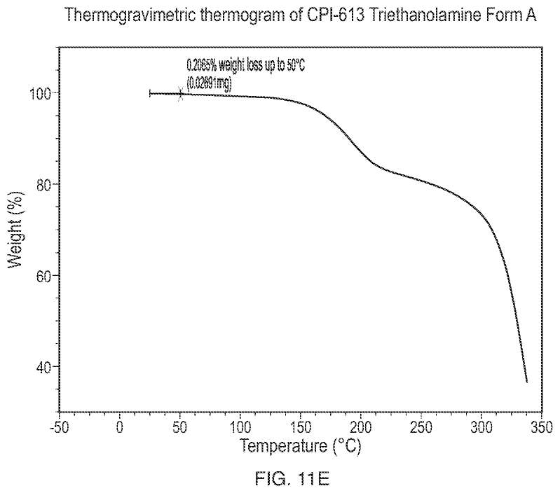

6,8-bis-benzylthio-octanoic acid (124.2 mg) and diethyl ether (0.5 mL) were charged to a glass vial. The slurry was contacted with a molar equivalent of triethanolamine (42.2 μL). The mixture was sonicated, generating a clear solution. The solution was contacted with 1 mL of heptane and sonicated forming a hazy suspension. The sample was evaporated under nitrogen leaving a clear oil. The oil was treated with 3 mL of heptane and noted to remain an oil. The sample was contacted with 3 mL of methyl tert-butyl ether and the oil viscosity increased. Seeds of CPI-613 triethanolamine form A were added. The sample was stored in a freezer (−25 to −10° C.) and observed the same day, where it was observed to have nucleated. Upon warming, apparent dissolution of the solids were observed. The sample was returned to the freezer condition for approximately 2 days before removal. The solution was decanted and the solids treated with approximately 20 mL of heptane and sonicated. The solids “flowed” but clumped as the sample warmed. The sample was returned to the freezer for an additional 8 days. The sample was removed from the freezer, the solution decanted, and the solids dried briefly under nitrogen. The sample was exposed to vacuum at ambient temperature for approximately 10 minutes. The final solids were composed of opaque fines and irregular birefringent blades as a crystalline, anhydrous salt with a 1:1 stoichiometry of 6,8-bis-benzylthio-octanoic acid to triethanolamine, denoted CPI-613 triethanolamine form A. Differential scanning calorimetry shows an endothermic event with an onset at 28° C. concurrent with a minor weight loss (0.2% weight loss up to 50° C.) by thermogravimetric analysis. The tentative unit cell parameters and calculated volume of CPI-613 triethanolamine form A at ambient temperature, derived from indexing, are: a=32.014 Å, b=5.768 Å, c=15.927 Å, α=90°, β=96.91°, γ=90°, V=2,919.7.0 Å³. The space group is P1 2₁/c (14). FIGS. 11A, 11B, 11C, 11D, and 11E present an X-ray powder diffraction pattern, differential scanning calorimetry thermogram, proton nuclear magnetic resonance spectrum, infrared spectroscopy, and thermogravimetric thermogram of CPI-613 triethanolamine form A. Peaks observed in the infrared spectrum of CPI-613 triethanolamine form A include 703, 752, 767, 777, 807, 847, 910, 970, 1011, 1032, 1058, 1069, 1102, 1155, 1203, 1241, 1261, 1294, 1326, 1347, 1398, 1451, 1479, 1493, 1569, 2854, 2921, and 3084 (all ±4 cm⁻¹). Peaks observed in the x-ray powder diffraction pattern of CPI-613 triethanolamine form A are listed in the table below.

| °2θ (±0.20) | d space (Å) | Intensity (%) |
|---|---|---|
| 2.76 | 31.961 ± 2.314 | 51 |
| 5.54 | 15.927 ± 0.574 | 11 |
| 8.33 | 10.600 ± 0.254 | 31 |
| 11.14 | 7.936 ± 0.142 | 11 |
| 11.87 | 7.447 ± 0.125 | 15 |
| 13.11 | 6.748 ± 0.103 | 48 |
| 13.92 | 6.356 ± 0.091 | 67 |
| 14.79 | 5.986 ± 0.081 | 14 |
| 16.42 | 5.394 ± 0.065 | 59 |
| 16.73 | 5.293 ± 0.063 | 42 |
| 17.48 | 5.069 ± 0.058 | 21 |
| 18.07 | 4.906 ± 0.054 | 13 |
| 19.02 | 4.662 ± 0.049 | 22 |
| 19.52 | 4.544 ± 0.046 | 70 |
| 20.23 | 4.385 ± 0.043 | 70 |
| 20.79 | 4.269 ± 0.041 | 18 |
| 21.37 | 4.155 ± 0.038 | 100 |
| 21.98 | 4.041 ± 0.036 | 30 |
| 22.37 | 3.972 ± 0.035 | 86 |
| 22.77 | 3.902 ± 0.034 | 66 |
| 23.04 | 3.858 ± 0.033 | 36 |
| 23.27 | 3.820 ± 0.032 | 20 |
| 23.94 | 3.713 ± 0.031 | 30 |
| 25.01 | 3.557 ± 0.028 | 26 |
| 26.42 | 3.371 ± 0.025 | 22 |
| 27.34 | 3.260 ± 0.023 | 14 |
| 28.07 | 3.177 ± 0.022 | 27 |
| 28.42 | 3.138 ± 0.022 | 13 |
| 28.97 | 3.080 ± 0.021 | 19 |

### Example 5—Spray Dried Dispersion Oral Formulation of 6,8-Bis-benzylthio-octanoic Acid

Solid amorphous dispersion formulations of 6,8-bis-benzylthio-octanoic acid (API) were prepared by mixing the API 1:4 with one of the following polymers: Eudragit L100, poly(vinylpyrrolidone) viscosity grade K30 (PVP K30), hydroxypropyl methyl cellulose (HPMC), cellulose acetate phthalate (CAP), or hydroxypropyl methylcellulose acetate succinate (HPMCAS-M), and spray drying from methanol or acetone using a small-scale Bend Lab Dryer with 35 kg/hr drying gas flow rate capacity (BLD-35). Conditions, yields, and residual solvent levels of two representative spray dried dispersion (SDD) formulations (75 g each) are presented in the following table.

| | 20% API: | 20% API: |
|---|---|---|
| Formulation | Eudragit L100 | HPMCAS-M |
| Spray Solution | 5% solids in methanol | 5% solids in acetone |
| Outlet Temp | 45° C. | 35° C. |
| Solution Feed Rate | 35 g/min | |
| Drying Gas Flow Rate | 475-500 g/min | |
| Atomization Pressure | 120 psi | |
| Nozzle | Schlick 2.0 pressure swirl atomizer | |
| Secondary Drying | 20 hr at 30° C. | |
| Dry Yield (%) | 94 | 96 |
| Residual Solvent (%) | 4.21 ± 0.02 (MeOH) | 1.01 ± 0.00 (Acetone) |
| (Wet SDD) | | |
| Residual Solvent (%) | <LOQ | <LOQ |
| (Tray-Dried Material) | | |
| API content by HPLC | 201 ± 1.1 mg/g | 198 ± 0.2 mg/g |

Scanning electron microscopy (SEM) was used to qualitatively determine particle morphology of the two SDD formulations, and to study if any degree of fusion or crystallinity was visually present. Particles show collapsed sphere morphology with no crystallization or fusion noted.

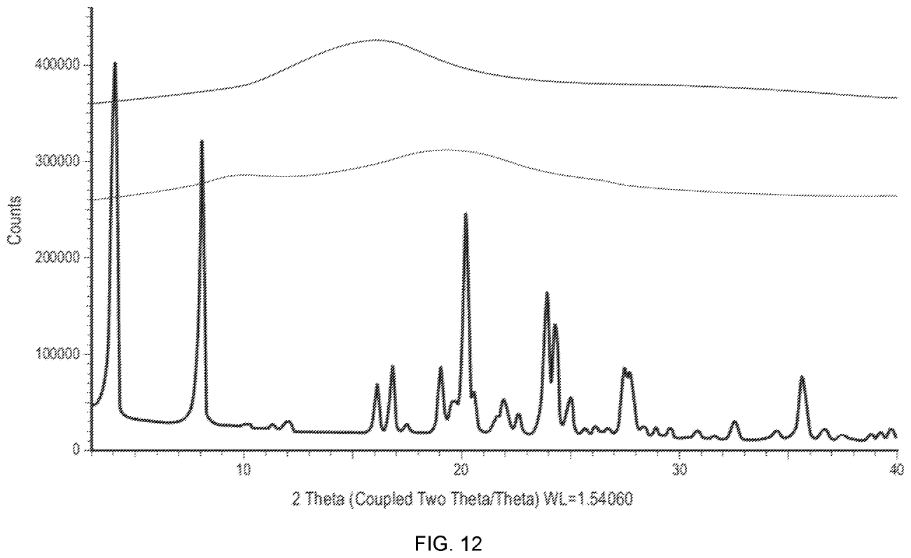

X-ray diffraction is typically sensitive to the presence of crystalline material with an LOD of about 1% of the sample mass. No crystallinity was detected by PXRD for either SDD formulation. Diffractograms in comparison to crystalline 6,8-bis-benzylthio-octanoic acid API can be found in FIG. 12, wherein the top diffractogram is the Eudragit L100 formulation, the middle diffractogram is the HPMCAS-M formulation, and the bottom diffractogram is crystalline 6,8-bis-benzylthio-octanoic acid.

### Example 6—Emulsion Oral Formulations of 6,8-Bis-benzylthio-octanoic Acid

Monolaurin (131 mg) and 6,8-bis-benzylthio-octanoic acid (93 mg) were warmed to 50° C. in polysorbate-80 (2.5 mL) in a round bottomed flask equipped with a magnetic stir bar. After complete dissolution to a clear solution, water (7.5 mL) was added with vigorous stirring at 50° C. to provide an emulsion.

6,8-bis-benzylthio-octanoic acid (312 mg) was combined with polysorbate 80 (6.25 g), soybean oil (1.25 g), and a lipid mix (100 mg) comprising cholesterol (14 g), cholesteryl acetate (14 g), cholesteryl benzoate (14 g), monolaurin (25.4 g), and monopalmitin (32.6 g), and the mixture heated to 50° C. until the solids dissolved (30 min). Dextrose (11.25 g) was dissolved in 236 mL of water, and the resulting aqueous dextrose solution was added to the oil solution above. The resulting two phase mixture was stirred for 30 min at rt, then vacuum filtered through a 0.22 um filter.

### Example 7—Liquid Formulations of 6,8-Bis-benzylthio-octanoic Acid

A 6,8-bis-benzylthio-octanoic acid solution was prepared by the steps of (a) providing a 50 mg/mL solution of 6,8-bis-benzylthio-octanoic acid in 1 M aqueous triethanolamine, and (b) diluting the 50 mg/mL solution with 5% aqueous dextrose to a concentration of 5 mg/mL. The resulting 5 mg/mL solution is identified as “7A” in Example 8 below.

A suspension vehicle was prepared by the steps of: (a) combining tris buffer (48 mg) and HPMCAS-HF (20 mg) in 14 mL of distilled water, (b) adjusting the pH to 7.4 with dilute sodium hydroxide to dissolve the HPMCAS-HF, (c) heating the resulting solution to approximately 90° C., (d) adding Methocel A4M Premium (100 mg) to the hot solution, (e) stirring the mixture vigorously to suspend the undissolved Methocel A4M, (f) cooling and stirring the mixture with an ice bath until the Methocel A4M dissolves (approximately 10 minutes), (g) diluting the solution with distilled/deionized water to bring the total volume to 20 mL, and (h) adjusting the pH to 7.4 with dilute acetic acid or dilute sodium hydroxide to provide the suspension vehicle.

Suspensions of the spray-dried formulations of Example 5 were prepared by adding 400 mg of the respective SDD formulation to a mortar, slowly adding 4 mL of the suspension vehicle (mixing thoroughly with a pestle after each small addition to uniformly disperse), and then transferring to a flask and stirring for one minute prior to administration. The resulting suspension of the Eudragit L100 SDD formulation (20 mg/mL 6,8-bis-benzylthio-octanoic acid) is identified as “7B” in Example 8 below. The resulting suspension of the HPMCAS-M SDD formulation (20 mg/mL 6,8-bis-benzylthio-octanoic acid) is identified as “7C” in Example 8 below.

In the same way, a 20 mg/mL suspension of 6,8-bis-benzylthio-octanoic acid was prepared by adding 80 mg 6,8-bis-benzylthio-octanoic acid to a mortar, slowly adding 4 mL of the suspension vehicle (mixing thoroughly with a pestle after each small addition to uniformly disperse), and then transferring to a flask and stirring for one minute prior to administration. The resulting suspension of 6,8-bis-benzylthio-octanoic acid is identified as “7D” in Example 8 below.

A solution of 6,8-bis-benzylthio-octanoic acid was prepared by dissolving SOLUTOL® (polyoxyl 15 hydroxystearate; KOLLIPHOR® HS 15) (3 grams) in distilled water (7 mL) to form a 30% solution, adding 6,8-bis-benzylthio-octanoic acid (50 mg) to 5 mL of the 30% solution, vortexing for 1 minute, and then sonicating for 45 minutes to provide a clear colorless solution (10 mg/mL; pH 7). The resulting solution is identified as “7E” in Example 8 below.

### Example 8—Oral Bioavailability of 6,8-Bis-benzylthio-octanoic Acid

Six groups of 16 BALB/c nude mice (8 males and 8 females) per group were administered 6,8-bis-benzylthio-octanoic acid in six different ways: (1) 5 μL/g IV injection (tail vein) of the triethanolamine/dextrose aqueous solution of Example 7 (25 mg/kg; 5 mL/kg; Ex. 7A); (2) 5 μL/g IP injection of the triethanolamine/dextrose aqueous solution of Example 7 (25 mg/kg; 5 mL/kg; 7A); (3) 5 μL/g oral administration of the Eudragit L100 SDD suspension of Example 7 (100 mg/kg; 5 mL/kg; 7B); (4) 5 μL/g oral administration of the HPMCAS-M SDD suspension of Example 7 (100 mg/kg; 5 mL/kg; 7C); (5) 5 μL/g oral administration of the 20 mg/mL 6,8-bis-benzylthio-octanoic acid suspension of Example 7 (100 mg/kg; 5 mL/kg; 7D); or (6) 10 μL/g oral administration of the 10 mg/mL SOLUTOL solution of Example 7 (100 mg/kg; 10 mL/kg; 7E). In each experiment, about 80 μL of blood was collected from one subgroup of 4 male and 4 female mice at 0.083, 1, 4, and 24 hours after dosing, and from the other subgroup of 4 male and 4 female mice at 0.5, 2, and 8 hours. Plasma from the collected blood samples was analyzed by LC-MS/MS for the presence of 6,8-bis-benzylthio-octanoic acid.

| | | | | Bio- | | | | |
|---|---|---|---|---|---|---|---|---|
| | | | Dose | avail- | AUC | | | T |
| | | Mice | (mg/ | ability | Last | Cmax | Tmax | ½ |
| Formulation | Route | (n) | kg) | (%) | (μM*hr) | (μM) | (hr) | (hr) |
| 7A (TEA/ | IV | 16 | 25 | — | 36 | 92 | 0.08 | 2.0 |
| dextrose) | | | | | | | | |
| 7A (TEA/ | IP | 16 | 25 | 83 | 29 | 103 | 0.08 | 3.9 |
| dextrose) | | | | | | | | |
| 7B (Eudragit | PO | 16 | 100 | 44 | 61 | 94 | 0.08 | 2.0 |
| SDD) | | | | | | | | |
| 7C (HPMCAS- | PO | 16 | 100 | 43 | 60 | 69 | 0.08 | 1.1 |
| MSDD) | | | | | | | | |
| 7D (CPI-613) | PO | 16 | 100 | 57 | 82 | 82 | 0.50 | 3.7 |
| 7E (Solutol) | PO | 16 | 100 | 127 | 175 | 229 | 0.08 | 4.4 |

This example demonstrates that 6,8-bis-benzylthio-octanoic acid is orally bioavailable.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating a disease or disorder in a patient in need thereof, comprising the step of orally administering to the patient a pharmaceutical composition comprising a therapeutically effective amount of 6,8-bis-benzylthio-octanoic acid or a pharmaceutically acceptable salt thereof, in order to treat the disease or disorder.

2. The method of claim 1, wherein the disease or disorder is cancer.

3. The method of claim 2, wherein the cancer is a lymphoma.

4. The method of claim 2, wherein the cancer is a leukemia.

5. The method of claim 2, wherein the cancer is a carcinoma.

6. The method of claim 2, wherein the cancer is a sarcoma.

7. The method of claim 2, wherein the cancer is a myeloma.

8. The method of claim 2, wherein the cancer is a brain or spinal cord cancer.

9. The method of claim 2, wherein the cancer is a melanoma.

10. The method of claim 2, wherein the cancer is a blastoma.

11. The method of claim 2, wherein the cancer is a germ cell tumor.

12. The method of claim 2, wherein the cancer is a cancer of the pancreas.

13. The method of claim 2, wherein the cancer is a cancer of the prostate.

14. The method of claim 3, wherein the lymphoma is relapsed or refractory Hodgkin lymphoma.

15. The method of claim 3, wherein the lymphoma is relapsed or refractory T-cell non-Hodgkin lymphoma.

16. The method of claim 3, wherein the lymphoma is relapsed or refractory Burkitt's lymphoma.

17. The method of claim 3, wherein the lymphoma is high-grade B-cell lymphoma with rearrangements of MYC and BCL2 and/or BCL6.

18. A method for delivering a therapeutically effective amount of 6,8-bis-benzylthio-octanoic acid to a patient in need thereof, comprising the step of orally administering to the patient a pharmaceutical composition comprising a therapeutically effective amount of 6,8-bis-benzylthio-octanoic acid or a pharmaceutically acceptable salt thereof.
